(19) United States
(12) Patent Application Publication
(10) Pub. No.: US 2026/0112493 A1
(43) Pub. Date: Apr. 23, 2026

(54) METHODS, DEVICES, AND SYSTEMS USING MULTIMODAL INPUT FOR CLINICAL APPLICATIONS

(71) Applicants: Yuanxu GAO, Weifang (CN); Kang ZHANG, Macao (CN)

(72) Inventors: Yuanxu GAO, Weifang (CN); Kang ZHANG, Macao (CN)

(21) Appl. No.: 19/414,783

(22) Filed: Dec. 10, 2025

Related U.S. Application Data
(63) Continuation of application No. PCT/CN2023/099559, filed on Jun. 10, 2023.

(51) Int. Cl. G16H 50/20; G06N 3/0464; G16H 10/60; G16H 30/40

(52) U.S. Cl. CPC G16H 50/20; G06N 3/0464; G16H 10/60; G16H 30/40

(57) ABSTRACT

Disclosed herein is a method for training a model comprising embedding data in a first modality in a first embedding layer to generate a sequence of first modality tokens, and embedding data in a second modality in a second embedding layer to generate a sequence of second modality tokens; passing the sequence of first modality tokens and the sequence of second modality tokens to a plurality of bidirectional multimodal attention blocks to generate a bag of unified tokens, wherein each bidirectional multimodal attention block applies attention to capture intramodal connections and intermodal connections, and wherein both the intramodal connections and the intermodal connections are encoded into latent representations, and representations within the same modality and across different modalities are learned and fused simultaneously.

### CROSS-REFERENCE TO RELATED APPLICATION

This application is a continuation of International Patent Application No. PCT/CN2023/099559, filed on Jun. 10, 2023, entitled “A TRANSFORMER-BASED REPRSENTATION-LEARNING MODEL WITH UNIFIED PROCESSING OF MULTIMODAL INPUT FOR CLINICAL DIAGNOSTICS AND PROGNOSTICS,” which application is herein incorporated by reference in its entirety for all purposes.

### FIELD

The present disclosure relates in some aspects to methods, devices, storage media, and systems involving unified processing of multimodal input for clinical diagnostics and prognostics, including in some aspects using a transformer-based representation-learning model.

### BRIEF SUMMARY

In some embodiments, disclosed herein are methods for providing a medical diagnosis for a patient, comprising: receiving one or more images of the patient and a set of text data associated with the patient; generating a plurality of tokens by: converting the one or more images into one or more visual tokens; and converting the set of text data into one or more textual tokens; obtaining the medical diagnosis of the patient by inputting the plurality of tokens into a trained machine learning model comprising a plurality of bidirectional blocks with intramodal intermodal attention; providing the medical diagnosis for the patient. In some embodiments, the medical diagnosis can comprise an identification of a disease or condition, a prediction of adverse clinical outcome, or a combination thereof. In any of the embodiments herein, the set of text data can comprise: narrative text, one or more text-field data, or a combination thereof. In any of the embodiments herein, the trained machine learning model can further comprise one or more self-attention blocks. In any of the embodiments herein, the trained machine learning model can further comprise a classification head.

In some embodiments, provided herein is a method for training a model for providing a medical diagnosis, wherein the model comprises a free-form embedding layer, an image embedding layer, a bidirectional multimodal attention block, a self-attention block, and a classification head, the method comprising (a) obtaining multimodal input data of a subject, wherein the multimodal input data comprise a set of clinical text data and a set of medical images. In some embodiments, the method further comprises (b) tokenizing and embedding the set of clinical text data in the free-form embedding layer to generate a sequence of clinical text tokens, and tokenizing and embedding the set of medical images in the image embedding layer to generate a sequence of image patch tokens. In some embodiments, the method further comprises (c) passing the sequence of clinical text tokens and the sequence of image patch tokens to the bidirectional multimodal attention block to generate a bag of unified tokens, wherein the bidirectional multimodal attention block applies (i) a first attention to capture intramodal connections in the set of clinical text data, (ii) a second attention to capture intramodal connections in the set of medical images, and (iii) a third attention to capture intermodal connections between the set of clinical text data and the set of medical images, and wherein both the intramodal connections and the intermodal connections are encoded into latent representations, and representations across modalities are learned and fused simultaneously. In some embodiments, the method further comprises (d) passing the bag of unified tokens to the self-attention block to learn a holistic multimodal representation of medical diagnosis for inputting to the classification head, thereby training the model. In some embodiments, a unified token herein is generated without distilling or exploiting common semantic information among different modalities.

In any of the embodiments herein including any preceding embodiment, the free-form embedding layer can be configured to convert unstructured and structured texts into uniform text tokens.

In any of the embodiments herein including any preceding embodiment, the free-form embedding layer can be configured to convert a chief complaint, a laboratory test result, and/or a demographic information into clinical text tokens.

In any of the embodiments herein including any preceding embodiment, the free-form embedding layer can comprise a dropout layer for clinical text tokens.

In any of the embodiments herein including any preceding embodiment, the image embedding layer can comprise a convolutional layer configured to produce a sequence of visual tokens.

In any of the embodiments herein including any preceding embodiment, the image embedding layer can comprise a dropout layer for image patch tokens.

In any of the embodiments herein including any preceding embodiment, the model can comprise two or more stacked bidirectional multimodal attention blocks.

In any of the embodiments herein including any preceding embodiment, each bidirectional multimodal attention block can comprise two-layer normalization layers.

In any of the embodiments herein including any preceding embodiment, the bidirectional multimodal attention block can comprise a bidirectional multimodal attention layer.

In any of the embodiments herein including any preceding embodiment, the bidirectional multimodal attention block can comprise a multilayer perceptron.

In any of the embodiments herein including any preceding embodiment, the model can comprise two or more stacked self-attention blocks.

In any of the embodiments herein including any preceding embodiment, the model can comprise ten self-attention blocks.

In any of the embodiments herein including any preceding embodiment, each self-attention block can comprise two-layer normalization layers.

In any of the embodiments herein including any preceding embodiment, each self-attention block can comprise a self-attention layer.

In any of the embodiments herein including any preceding embodiment, each self-attention block can comprise a multilayer perceptron.

In any of the embodiments herein including any preceding embodiment, the classification head can be configured to identify a disease or condition in a patient.

In any of the embodiments herein including any preceding embodiment, the classification head can be configured to predict an adverse clinical outcome in a patient.

In any of the embodiments herein including any preceding embodiment, the set of clinical text data can comprise a chief complaint, a demographic information, and a laboratory test report.

In any of the embodiments herein including any preceding embodiment, the chief complaint can comprise unstructured data.

In any of the embodiments herein including any preceding embodiment, the chief complaint can comprise structured data.

In any of the embodiments herein including any preceding embodiment, the set of clinical text data can comprise: an unstructured chief complaint comprising a history of present and past illness; a comorbidity; a symptom; gender; and/or age.

In any of the embodiments herein including any preceding embodiment, the set of medical images can comprise one or more CT images, one or more X-ray images, one or more optical coherence tomography (OCT) images, one or more retinal fundus photographs, one or more fundus fluorescein angiography (FFA) images, one or more indocyanine green angiography (ICGA) images, or a combination thereof.

In any of the embodiments herein including any preceding embodiment, in (b), encoded feature vectors for each category of data in the set of clinical text data can be concatenated to produce the sequence of clinical text tokens.

In any of the embodiments herein including any preceding embodiment, in (c), the bidirectional multimodal attention block can comprise multiple heads configured to perform attention operations in multiple representation subspaces simultaneously and aggregate results from the multiple representation subspaces afterwards.

In any of the embodiments herein including any preceding embodiment, in (d), the self-attention block can comprise multiple heads configured to perform attention operations in multiple representation subspaces simultaneously and aggregate results from the multiple representation subspaces afterwards.

In any of the embodiments herein including any preceding embodiment, the method can comprise applying average pooling to the bag of unified tokens generated from the self-attention block.

In some embodiments, disclosed herein is a method for training a model for providing a medical diagnosis, wherein the model comprises a first embedding layer, a second embedding layer that differs from the first embedding layer, a bidirectional multimodal attention block, a self-attention block, and a classification head, the method comprising: (a) obtaining multimodal input data of a subject, wherein the multimodal input data comprise data in a first modality and data in a second modality which differs from the first modality. In some embodiments, the method comprises (b) tokenizing and embedding the data in the first modality in the first embedding layer to generate a sequence of first modality tokens, and tokenizing and embedding the data in the second modality in the second embedding layer to generate a sequence of second modality tokens. In some embodiments, the method comprises (c) passing the sequence of first modality tokens and the sequence of second modality tokens to the bidirectional multimodal attention block to generate a bag of unified tokens, wherein the bidirectional multimodal attention block applies (i) a first attention to capture intramodal connections in the data in the first modality, (ii) a second attention to capture intramodal connections in the data in the second modality, and (iii) a third attention to capture intermodal connections between the data in the first modality and the data in the second modality, and wherein both the intramodal connections and the intermodal connections are encoded into latent representations, and representations across modalities are learned and fused simultaneously. In some embodiments, the method comprises (d) passing the bag of unified tokens to the self-attention block to learn a holistic multimodal representation of medical diagnosis for inputting to the classification head, thereby training the model.

In any of the embodiments herein including any preceding embodiment, the first modality can comprise text data.

In any of the embodiments herein including any preceding embodiment, the data in the first modality can comprise unstructured data.

In any of the embodiments herein including any preceding embodiment, the second modality can comprise image data.

In some embodiments, provided herein is a transformer model comprising a free-form embedding layer, an image embedding layer, a plurality of bidirectional multimodal attention blocks, a self-attention block, and a classification head. In some embodiments, provided herein is a transformer model comprising a free-form embedding layer, an image embedding layer, a plurality of bidirectional multimodal attention blocks, a plurality of self-attention blocks, and a classification head. In any of the embodiments herein including any preceding embodiment, the model can be trained by forwarding multimodal input data of a patient, wherein the multimodal input data comprise a set of medical images and a set of clinical text data, to the free-form embedding layer and the image embedding layer, where the set of medical images are tokenized and embedded to generate a sequence of image patch tokens, and the set of clinical text data are tokenized and embedded to generate a sequence of clinical text tokens. In any of the embodiments herein including any preceding embodiment, the sequence of clinical text tokens and the sequence of image patch tokens can be passed to the plurality of bidirectional multimodal attention blocks to generate a bag of unified tokens. In any of the embodiments herein including any preceding embodiment, each of the plurality of bidirectional multimodal attention blocks can apply (i) a first attention to capture intramodal connections in the set of clinical text data, (ii) a second attention to capture intramodal connections in the set of medical images, and (iii) a third attention to capture intermodal connections between the set of clinical text data and the set of medical images. In any of the embodiments herein including any preceding embodiment, both the intramodal connections and the intermodal connections can be encoded into latent representations, and representations within the same modality and across multiple modalities can be learned and fused simultaneously. In any of the embodiments herein including any preceding embodiment, the bag of unified tokens can be passed to the self-attention block to learn a holistic multimodal representation of medical diagnosis for inputting to the classification head, thereby training the model for providing the medical diagnosis for the patient.

In any of the embodiments herein including any preceding embodiment, the method can be independent of the distillation and exploitation of common semantic information among different modalities to provide supervision for model training.

In some embodiments, disclosed herein is a method of generating a medical diagnosis for a subject, the method comprising: receiving a prompt for obtaining the medical diagnosis and a set of data related to the subject, and generating the medical diagnosis by inputting the prompt and the set of data in a trained model generated by the method of any embodiment disclosed herein including any preceding embodiment.

In any of the embodiments herein including any preceding embodiment, the medical diagnosis can comprise identification of a pulmonary disease or condition in the subject.

In any of the embodiments herein including any preceding embodiment, the medical diagnosis can comprise prediction of an adverse clinical outcome in the subject.

In any of the embodiments herein including any preceding embodiment, the method can be a computer-implemented method.

In some embodiments, provided herein is a system comprising: at least one hardware processor; and one or more software modules configured to, when executed by the at least one hardware processor, perform the method of any embodiment disclosed herein including any preceding embodiment.

In some embodiments, provided herein is a non-transitory computer-readable medium having instructions stored thereon, wherein the instructions, when executed by a processor, cause the processor to perform the method of any embodiment disclosed herein including any preceding embodiment.

In some embodiments, provided herein is a system comprising: at least one hardware processor; non-transitory computer-readable medium coupled to at least one hardware processor, optionally wherein the coupling is over a network; and instructions stored in the non-transitory computer-readable medium, wherein the instructions when implemented by the processor, configure the system to perform the method of any embodiment disclosed herein including any preceding embodiment.

### BRIEF DESCRIPTION OF THE DRAWINGS

The drawings illustrate certain features and advantages of this disclosure. These embodiments are not intended to limit the scope of the appended claims in any manner. Various aspects of the disclosed methods, devices, and systems are set forth with particularity in the appended claims, and an understanding of the features and advantages of the disclosed methods, devices, and systems will be obtained by reference to the following detailed description of illustrative embodiments and the accompanying drawings, of which:

FIGS. 1A-1E introduce a unified AI-based medical diagnostic model designed to make decisions by jointly learning holistic representations of medical images, unstructured chief complaint and structured clinical information. FIG. 1A contrasts a previous non-unified multimodal diagnosis paradigm with the unified model, which eliminates the tedious text structuralization process, separate paths for modality-specific feature extraction, and the multimodal feature fusion module in traditional non-unified approaches. Instead, the unified model performs multimodal diagnosis with a single, unified transformer. FIG. 1B shows the scheme for splitting an original dataset into training, validation and testing sets for pulmonary disease identification and adverse clinical outcome prediction of COVID-19, respectively. FIG. 1C compares the experimental results from the image-only models, non-unified early fusion methods, multimodal transformer (Perceiver), and the unified model in pulmonary disease identification. FIG. 1D compares the experimental results from the image-only models, non-unified early fusion methods, multimodal transformer (Perceiver), and the unified model in adverse clinical outcome prediction of COVID-19. p-values were calculated between the mean performance of the models using the independent two-sample t-test (two-sided). Specifically, each experiment was repeated for ten times with different random seeds, after which p-values were calculated. FIG. 1E compares the unified model with junior (with <7 years of experience, n=2) and senior physicians (with more than 7 years of experience, n=2), where average performance within each group was reported. The unified model surpassed the diagnosis performance of junior physicians while performing competitively with senior experts. AUC, area under the curve.

FIGS. 2A-2F depict the network architecture of the unified model. FIG. 2A shows the overall workflow of the unified model in a first task, pulmonary disease identification. The input data consist of five parts: the chief complaint (ChiComp), laboratory-test results (LabTest), demographics (sex and age), and radiograph. The multimodal diagnosis transformer (MDT) includes two bi-directional multimodal attention blocks and ten self-attention blocks. The training process is guided by pulmonary disease annotations provided by human experts. FIG. 2B demonstrates the encoding of different types of clinical texts in the free-form embedding. Specifically, the unified model accepts unstructured chief complaints as part of the input. FIG. 2C shows encoding a radiograph as a sequence of image patch tokens. FIG. 2D presents the detailed design of a bi-directional multimodal attention block, which contains two-layer normalization layers (Norm), a bi-directional multimodal attention layer and a multi-layer perceptron (MLP). FIG. 2E presents detailed attention operations in the bi-directional multimodal attention layer, where representations within the same modality (e.g., Q_{T} to two matrix multiplications (mat. mul.) within the clinical text data modality, and Q_{I} to two matrix multiplications within the image data modality) and across multiple modalities (e.g., K_{T} to one matrix multiplication within the clinical text data modality and one matrix multiplication within the image data modality, K_{I} to one matrix multiplication within the clinical text data modality and one matrix multiplication within the image data modality; and V_{T} to one matrix multiplication within the clinical text data modality and one matrix multiplication within the image data modality, V_{I} to one matrix multiplication within the clinical text data modality and one matrix multiplication within the image data modality) are learned and fused simultaneously. FIG. 2F shows the detailed architecture of a self-attention block. PI, position injection.

FIGS. 3A-3F depict an attention analysis. FIG. 3A presents the attention allocated to different types of inputs from a patient with COPD, including the radiograph, chief complaint (ChiComp), laboratory-test results (LabTest), and demographics. FIG. 3B shows the relative importance of laboratory-test items. FIG. 3C compares the importance of sex and age in making a diagnostic decision. FIG. 3D visualizes the attention assigned to individual pixels in the radiograph. The left figure is the input chest X-ray. The right figure presents pixels with different attention values. FIG. 3E shows the impact of cross attention on the relevance and importance of high-ranking words (from chief complaints) and image patches (from radiographs) in the pulmonary disease identification task. Specifically, high-ranking words and patches are defined as those whose tokens have top 25% cosine similarity scores with the CLS token. FIG. 3F presents the normalized importance of every word in the chief complaint, with visualization showing the distribution of attention between every image patch and each of the top 3 ranked words. The color bars in the radiographs illustrates the unified model's confidence about a pixel being abnormal, where a bright color stands for high confidence, and a dark color denotes low confidence.

FIG. 4A depicts impact of chief complaints on each respiratory disease. FIG. 4B depicts impact of laboratory-test results on each respiratory disease. Either the chief complaint or the laboratory test results were removed from the input and the performance drop on each disease was reported using AUROC as an evaluation metric.

### DETAILED DESCRIPTION

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

During the diagnostic process, clinicians leverage multimodal information, such as the chief complaint, medical images and laboratory test results. Deep-learning models for aiding diagnosis have yet to meet this requirement of leveraging multimodal information. In some embodiments, disclosed herein is a transformer-based representation-learning model as a clinical diagnostic aid that processes multimodal input in a unified manner. Rather than learning modality-specific features, the model leverages embedding layers to convert images and unstructured and structured text into visual tokens and text tokens, and uses bidirectional blocks with intramodal and intermodal attention to learn holistic representations of radiographs, the unstructured chief complaint and clinical history, and structured clinical information such as laboratory test results and patient demographic information. In some embodiments, the unified model outperforms an image-only model and non-unified multimodal diagnosis models, for instance, in the identification of disease and/or in the prediction of adverse clinical outcomes in patients, thereby streamlining the triaging of patients and facilitating the clinical decision-making process.

It has been a common practice in modern medicine to utilize multimodal clinical information for medical diagnosis. For instance, apart from chest radiographs, thoracic physicians need to take into account each patient's demographics (e.g., age and gender), the chief complaint (e.g., history of present and past illness), and the laboratory-test report to make accurate diagnostic decisions. In practice, abnormal radiographic patterns are first associated with symptoms mentioned in the chief complaint or abnormal results in the laboratory-test report. Then, physicians rely on their rich domain knowledge and years of training to make optimal diagnoses by jointly interpreting such multimodal data^{1,2}. The importance of exploiting multimodal clinical information has been extensively verified in the literature³⁻¹⁰ in different specialties, including but not limited to, radiology, dermatology, and ophthalmology.

The above multimodal diagnostic workflow requires enormous expertise, which may not be available in geographic regions with limited medical resources. Meanwhile, simply increasing the workload of experienced physicians and radiologists would inevitably exhaust their energy and thus increase the risk of misdiagnosis. To meet the increasing demand for precision medicine, machine learning techniques¹¹ have become the de facto choice for automatic yet intelligent medical diagnosis. Among them, the unprecedented development of deep learning^{12,13} endows machine learning models with the ability to detect diseases from medical images near or at the level of human experts¹⁴⁻¹⁸.

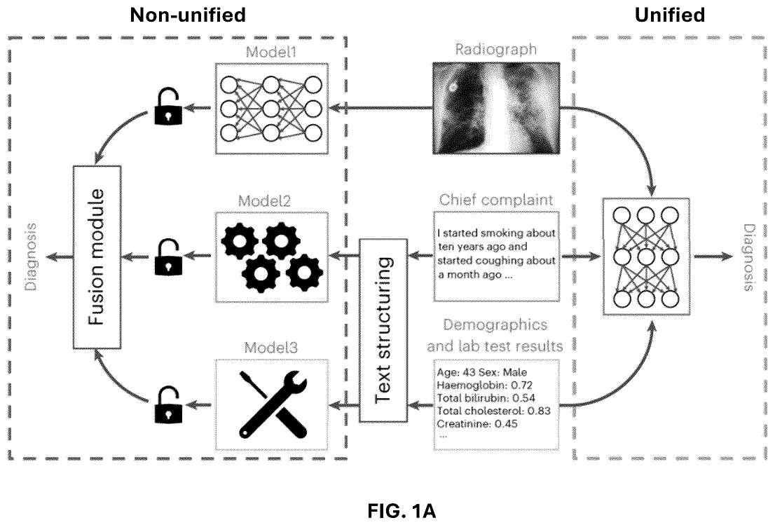

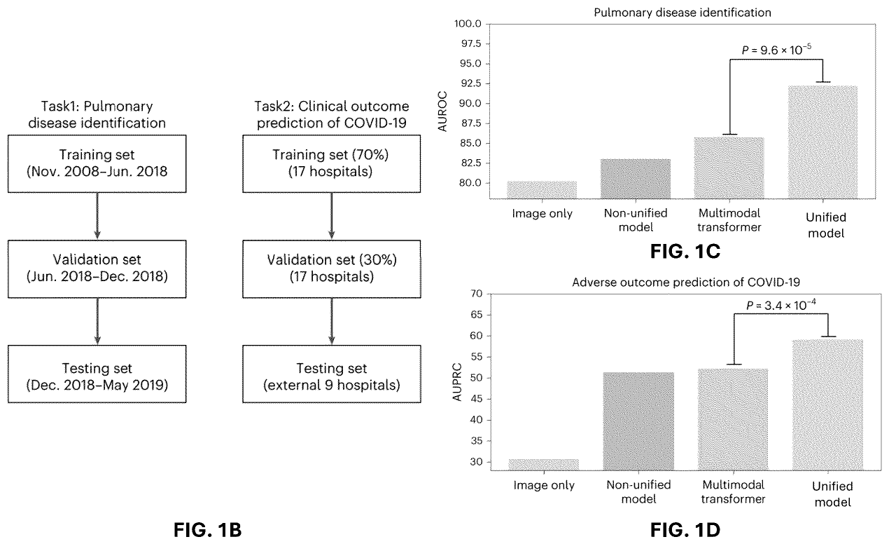

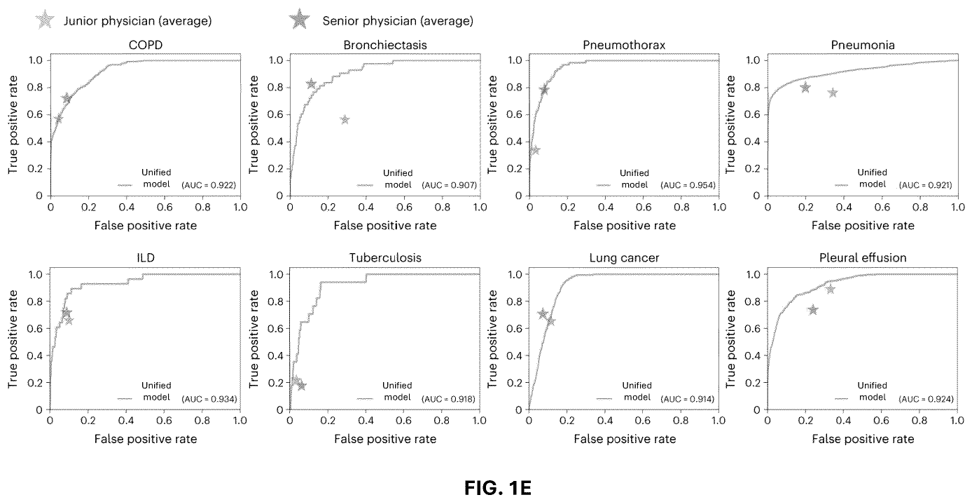

Although AI-based medical image diagnosis has achieved tremendous progress in recent years, it is still debatable how to jointly interpret medical images and their associated clinical context. As illustrated in FIG. 1A, current multimodal clinical decision support systems¹⁹⁻²³ mostly lean upon a non-unified way to fuse information from multiple sources. Given a set of input data from different sources, these approaches first roughly divide them into three basic modalities, i.e., images, narrative text (e.g., the chief complaint that includes the history of present and past illness), and structured fields (e.g., demographics and laboratory-test results). Next, a text structuralization process is introduced to transform the narrative text into structured tokens. Then, data in different modalities are fed to different machine learning models to produce modality-specific features or predictions. Finally, a fusion module is employed to unify these modality-specific features or predictions for making final diagnostic decisions. In practice, according to whether joining multiple input modalities at the feature or prediction level, these non-unified methods can be further categorized into early¹⁹⁻²² or late fusion²³ methods.

One issue of early and late fusion methods is that they separate the multimodal diagnostic process into two relatively independent stages: modality-specific model training and diagnosis-oriented fusion. However, such a design has one obvious limitation: the inability to encode the connections and associations among different modalities. Another drawback of these non-unified approaches lies in the text structuralization process, which is cumbersome and still labor-intensive, even with the assistance of modem natural language processing (NLP) tools. On the other hand, transformer-based architectures²⁴ are poised to broadly reshape natural language processing²⁵ and computer vision²⁶. Compared to convolutional neural networks²⁷ and word embedding algorithms^{28,29}, transformers²⁴ impose few assumptions about the input data form and thus have the potential to learn higher-quality feature representations from multimodal input data. More importantly, the basic architectural component in transformers (i.e., the self-attention block) remains nearly unchanged across different modalities^{25,26}, providing an opportunity to build a unified yet flexible model to conduct representation learning on multimodal clinical information.

In some embodiments, disclosed herein is a unified AI-based medical diagnostic model designed to make decisions by jointly learning holistic representations of multimodal input, for example, including medical images, unstructured chief complaint, and structured clinical information. In some embodiments, provided herein is a method of providing a medical diagnosis for a patient, comprising: receiving one or more images of the patient and a set of text data associated with the patient; generating a plurality of tokens by: converting the one or more images into one or more visual tokens; and converting the set of text data into one or more textual tokens; obtaining the medical diagnosis of the patient by inputting the plurality of tokens into a trained machine learning model comprising a plurality of bidirectional blocks with intramodal and intermodal attention; providing the medical diagnosis for the patient. In some embodiments, the medical diagnosis comprises an identification of a disease, a prediction of adverse clinical outcome, or a combination thereof. In some embodiments, the set of text data comprises: narrative text, one or more text-field data, or a combination thereof. In some embodiments, the trained machine learning model further comprises one or more self-attention blocks. In some embodiments, the trained machine learning model further comprises a classification head.

In some embodiments, disclosed herein is a method for training a model comprising a first embedding layer, a second embedding layer that differs from the first embedding layer, a plurality of bidirectional multimodal attention blocks, a self-attention block, and a classification head. In some embodiments, the method comprises embedding data in a first modality in the first embedding layer to generate a sequence of first modality tokens, and embedding data in a second modality in the second embedding layer to generate a sequence of second modality tokens; passing the sequence of first modality tokens and the sequence of second modality tokens to the plurality of bidirectional multimodal attention blocks to generate a bag of unified tokens, wherein each bidirectional multimodal attention block applies (i) a first attention to capture intramodal connections in the data in the first modality, (ii) a second attention to capture intramodal connections in the data in the second modality, and (iii) a third attention to capture intermodal connections between the data in the first modality and the data in the second modality, and wherein both the intramodal connections and the intermodal connections are encoded into latent representations, and representations within the same modality and across different modalities are learned and fused simultaneously; and passing the bag of unified tokens to the self-attention block to learn a holistic multimodal representation for inputting to the classification head, thereby training the model.

In some embodiments, provided herein is a method for training a model for providing a medical diagnosis, wherein the model comprises a first embedding layer, a second embedding layer that differs from the first embedding layer, a bidirectional multimodal attention block, a self-attention block, and a classification head, the method comprising: (a) obtaining multimodal input data of a subject, wherein the multimodal input data comprise data in a first modality and data in a second modality which differs from the first modality; (b) tokenizing and embedding the data in the first modality in the first embedding layer to generate a sequence of first modality tokens, and tokenizing and embedding the data in the second modality in the second embedding layer to generate a sequence of second modality tokens; (c) passing the sequence of first modality tokens and the sequence of second modality tokens to the bidirectional multimodal attention block to generate a bag of unified tokens, wherein the bidirectional multimodal attention block applies (i) a first attention to capture intramodal connections in the data in the first modality, (ii) a second attention to capture intramodal connections in the data in the second modality, and (iii) a third attention to capture intermodal connections between the data in the first modality and the data in the second modality, and wherein both the intramodal connections and the intermodal connections are encoded into latent representations, and representations across modalities are learned and fused simultaneously; and (d) passing the bag of unified tokens to the self-attention block to learn a holistic multimodal representation of medical diagnosis for inputting to the classification head, thereby training the model.

In some embodiments, disclosed herein is a unified AI-based medical diagnostic model designed to make decisions by jointly learning holistic representations of medical images, unstructured chief complaint, and structured clinical information. In some embodiments, the model is a single, unified AI model to conduct holistic representation learning on multimodal clinical information simultaneously, an example of which is shown in FIG. 1A. In some embodiments, the model comprises a unified multimodal diagnostic transformer (MDT) and bi-directional multimodal attention blocks. In some embodiments, the unified model comprises a transformer stack that directly produces diagnostic results from multimodal input data, enabling the model to take a different approach from previous non-unified methods by learning holistic representations from multimodal clinical information progressively while eliminating separate paths for learning modality-specific features. In addition, using the unified multimodal diagnostic transformer, the model is able to perform representation learning on top of unstructured raw text, which avoids tedious text structuralization steps in non-unified approaches. For better handling the differences among modalities, the model introduces bi-directional multimodal attention to bridge the gap between token-level modality-specific features and high-level diagnosis-oriented holistic representations by explicitly encoding the interconnections among different modalities, which explicit encoding process can be regarded as a complement to the holistic multimodal representation learning process in MDT.

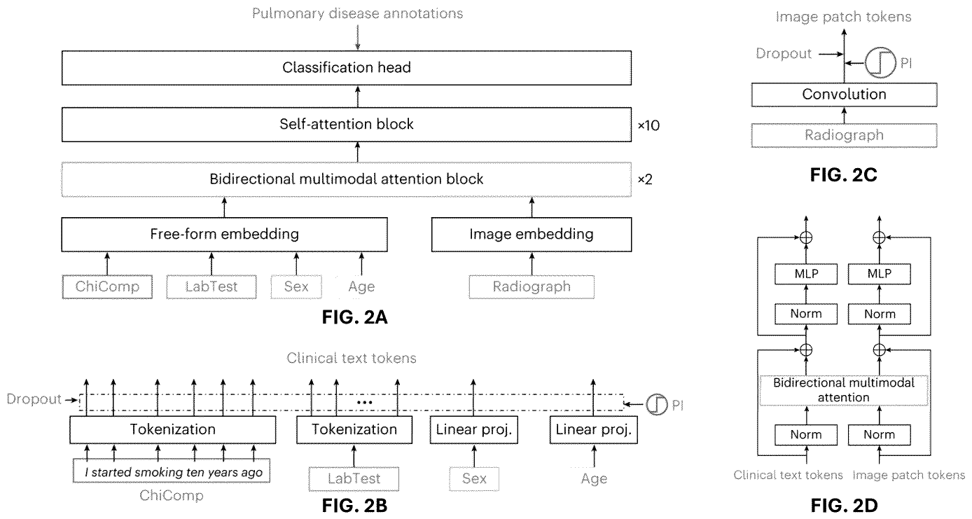

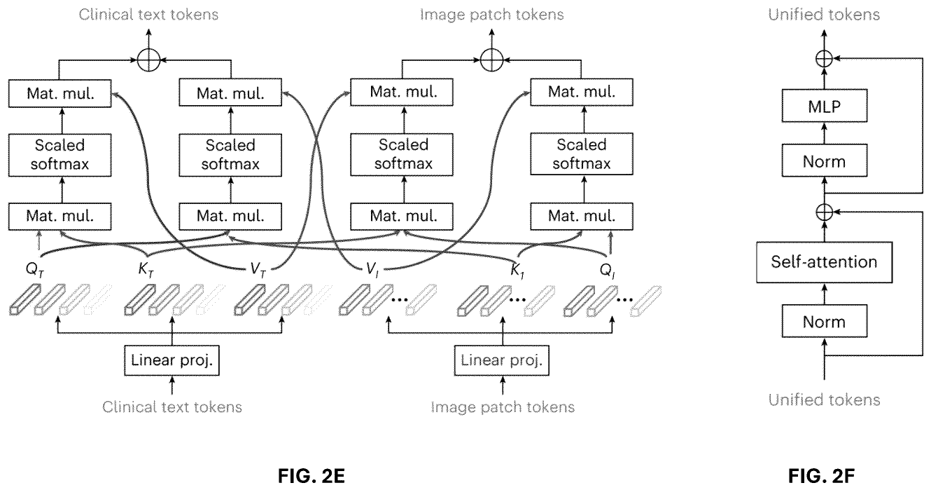

As shown in FIG. 2A, the unified multimodal diagnostic transformer is primarily composed of embedding layers, bi-directional multimodal blocks, and self-attention blocks. In some embodiments, the unified multimodal diagnostic transformer allows the model to jointly interpret multimodal clinical information simultaneously. Specifically, a free-form embedding layer is employed to convert unstructured and structured texts into uniform text tokens, an example of which is shown in FIG. 2B. Meanwhile, a similar tokenization procedure is also applied to each input image (example shown in FIG. 2C). In some embodiments, two bi-directional multimodal blocks (example shown in FIG. 2D) are stacked to learn fused mid-level representations across multiple modalities. In addition to computing intra-modal attention among tokens from the same modality, these blocks also explicitly compute inter-modal attention among tokens across different modalities (example shown in FIG. 2E). Thus, in some embodiments, the unified model bridges the gap between token-level modality-specific features and high-level diagnosis-oriented holistic representations by explicitly encoding the interconnections among different modalities. In some embodiments, the attention operations in the bi-directional multimodal attention layer, not only representations within the same modality are learned and fused, but also representations across multiple modalities are learned and fused simultaneously, thereby encoding the interconnections among different modalities. For instance, as shown in FIG. 2E, the attention operation connects Q_{T} to two matrix multiplications (mat. mul.) within the clinical text data modality, and Q₁ to two matrix multiplications within the image data modality. At the same time, the attention operation connects K_{T} to one matrix multiplication within the clinical text data modality and one matrix multiplication within the image data modality; connects K₁ to one matrix multiplication within the clinical text data modality and one matrix multiplication within the image data modality; connects V_{T} to one matrix multiplication within the clinical text data modality and one matrix multiplication within the image data modality; and connects V₁ to one matrix multiplication within the clinical text data modality and one matrix multiplication within the image data modality.

These intra- and inter-modal attentional operations are consistent with daily clinical practices, where physicians need to discover interconnected information within the same modality as well as across different modalities. In reality, these connections are often hidden among local patterns, such as words in the chief complaint and image regions in radiographs, and different local patterns may refer to the same lesion or the same disease. Therefore, such connections provide mutual confirmations of clinical evidence and are helpful to both clinical and AI-based diagnosis. In bi-directional multimodal attention, each token can be regarded as the representation of a local pattern, and token-level intra- and inter-modal attention respectively capture the interconnections among local patterns from the same modality and across different modalities. In comparison, previous non-unified methods make diagnoses on top of separate global representations of input data in different modalities and thus cannot exploit the underlying local interconnections. In some embodiments, self-attention blocks are stacked (example shown in FIG. 2F) to learn multimodal representations.

Some recent vision-language fusion approaches³¹⁻³³ heavily rely on the distillation and exploitation of common semantic information among different modalities to provide supervision for model training. In some embodiments, a unified model disclosed herein aims to learn a joint multimodal representation. In some embodiments, a unified model disclosed herein differs from a vision-language fusion model in the roles of the different modalities. In some embodiments, a unified model disclosed herein is designed for the scenario where multiple modalities supply complementary semantic information, which can be fused and utilized to improve prediction performance. In some embodiments, a unified model disclosed herein does not rely on the distillation and exploitation of common semantic information among different modalities to provide supervision for model training. In some embodiments, a unified token herein (e.g., as shown in FIG. 2F) is generated without distilling or exploiting common semantic information among different modalities.

In some embodiments, a method disclosed herein comprises collecting medical images such as radiographs (e.g., CXR images), for instance, as part of the patients' routine clinical care. In some embodiments, the method comprises using the collected medical images for training a unified model disclosed herein for identifying a disease or condition, e.g., for pulmonary disease identification. In some embodiments, a medical image (e.g., a radiograph such as an CXR image) is first de-identified to remove any patient-related information. In some embodiments, the collected CXR images comprise both anterior views and posterior views of CXR images.

In some embodiments, a method disclosed herein comprises collecting clinical data including for example textual clinical data. In some embodiments, the textual clinical data comprise one or more types of textual clinical data. In some embodiments, the textual clinical data comprise one or any combination of a chief complaint, a demographic information, and a laboratory-test result. In some embodiments, the textual clinical data comprise one or any combination of a unstructured chief complaint, a demographic information, and a laboratory-test result. In some embodiments, the textual clinical data comprise one or any combination of a chief complaint comprising history of present and past illness, age and/or gender, and a laboratory-test result. In some embodiments, the chief complaint is unstructured. In some embodiments, the demographics and laboratory-test results are structured.

In some embodiments, the chief complaint is an unstructured chief complaint comprising history of present and past illness. In some embodiments, the maximum length of the chief complaint is about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, or more than 200 words. In some embodiments, the maximum length of the chief complaint is 40 or fewer words. In some embodiments, when a patient's chief complaint has more than 40 words, only the first 40 words are taken; otherwise, zero padding can be used to satisfy the length requirement.

In some embodiments, a laboratory-test comprises one or more selected from the group consisting of magnesium, alanine aminotransferase, urea, serum inorganic phosphorus, serum beta hydroxybutyric acid assay, indirect bilirubin, high density lipoprotein, direct bilirubin, portaline aminotransferase, serum cystatin C assay, globulin, lactate dehydrogenase, AST/ALT, creatine kinase, calcium, glucose, creatinine, carbon dioxide binding capacity, potassium, total bilirubin, chlorine, hydroxybutyrate dehydrogenase, cholesterol, white globule ratio, glutamyl transpeptidase, albumin, total protein, anion gap, uric acid, triglycerides, alkaline phosphatase, low density lipoprotein, sodium, PaCO₂, PaO₂, triglycerides, microprotein, adenosine deaminase, estimated glomerular filtration rate, total bile acids, urinary sodium/urinary creatinine, urinary potassium/urinary creatinine, cholinesterase, urinary calcium/urinary creatinine, urinary magnesium/urinary creatinine, 24-hour urine volume, urinary phosphorus/urinary creatinine, apolipoprotein A1, urine chlorine/urine creatinine, A1/B100, apolipoprotein B100, lipoprotein(a), homocysteine, eGFR gender index, cystatin C, phosphorus, gamma glutamyl transpeptidase, oxygen saturation, calcium ion, corrected ionized calcium, calculated erythrocyte pressure value, whole blood base residual, oxygen partial pressure, extracellular fluid alkaline residual, pH, partial pressure of carbon dioxide, body temperature, total carbon dioxide, standard bicarbonate concentration, bicarbonate, total haemoglobin concentration, buffer bases, atmospheric pressure, haemoglobin half-saturation and oxygen partial pressure, reduced haemoglobin, FIO₂, high iron haemoglobin, oxygen content, carboxyhaemoglobin, oxyhaemoglobin concentration, oxygen volume, haemoglobin, respiratory index, corrected pH, HCO₃ concentration, whole blood lactate, total carbon dioxide, blood glucose, lipase, amylase, uranitin, and parathyroid hormone. In some embodiments, each patient's laboratory-test report contains one or more results from a blood test, for example, any one or more of magnesium, alanine aminotransferase, urea, serum inorganic phosphorus, serum beta hydroxybutyric acid assay, indirect bilirubin, high density lipoprotein, direct bilirubin, portaline aminotransferase, serum cystatin C assay, globulin, lactate dehydrogenase, AST/ALT, creatine kinase, calcium, glucose, creatinine, carbon dioxide binding capacity, potassium, total bilirubin, chlorine, hydroxybutyrate dehydrogenase, cholesterol, white globule ratio, glutamyl transpeptidase, albumin, total protein, anion gap, uric acid, triglycerides, alkaline phosphatase, low density lipoprotein, sodium, PaCO₂, PaO₂, triglycerides, microprotein, adenosine deaminase, estimated glomerular filtration rate, total bile acids, urinary sodium/urinary creatinine, urinary potassium/urinary creatinine, cholinesterase, urinary calcium/urinary creatinine, urinary magnesium/urinary creatinine, 24-hour urine volume, urinary phosphorus/urinary creatinine, apolipoprotein AI, urine chlorine/urine creatinine, A1/B100, apolipoprotein B100, lipoprotein(a), homocysteine, eGFR gender index, cystatin C, phosphorus, gamma glutamyl transpeptidase, oxygen saturation, calcium ion, corrected ionized calcium, calculated erythrocyte pressure value, whole blood base residual, oxygen partial pressure, extracellular fluid alkaline residual, pH, partial pressure of carbon dioxide, body temperature, total carbon dioxide, standard bicarbonate concentration, bicarbonate, total haemoglobin concentration, buffer bases, atmospheric pressure, haemoglobin half-saturation and oxygen partial pressure, reduced haemoglobin, FIO₂, high iron haemoglobin, oxygen content, carboxyhaemoglobin, oxyhaemoglobin concentration, oxygen volume, haemoglobin, respiratory index, corrected pH, HCO₃ concentration, whole blood lactate, total carbon dioxide, blood glucose, lipase, amylase, uranitin, and parathyroid hormone. In some embodiments, a laboratory test result is normalized through min-max scaling so that a normalized value lies in [0, 1], where the minimum and maximum values in min-max scaling are determined using the training set. In some embodiments, −1 is used to denote missing values. In some embodiments, a laboratory-test is a test for respiratory-disease identification.

In some embodiments, the clinical data comprises a demographic information, a chief complaint, and/or a laboratory-test result. In some embodiments, the clinical data comprises a demographic information, a structured chief complaint, and/or a laboratory-test result. In some embodiments, the clinical data comprises age and/or gender; a comorbidity; a symptom; and/or a laboratory test result. In some embodiments, the clinical data comprises age and gender; comorbidities; symptoms; and laboratory test results. In some embodiments, the clinical data comprises a structured chief complaint comprising a comorbidity and/or a symptom. In some embodiments, the clinical data comprises a structured chief complaint consisting of comorbidities and symptoms. In some embodiments, median imputation is applied to fill in missing values. In some embodiments, the clinical data comprises one or more comorbidities selected from the group consisting of coronary heart disease, diabetes, hypertension, chronic obstructive lung disease (COPD), chronic liver disease, chronic kidney disease, and carcinoma. In some embodiments, the clinical data comprises one or more clinical symptoms selected from the group consisting of fever, cough, myalgia, fatigue, headache, nausea or vomiting, diarrhoea, abdominal pain, and dyspnoea. In some embodiments, the clinical data comprises one or more laboratory results selected from the group consisting of white blood cell, neutrophil, lymphocyte, platelet, haemoglobin, prothrombin time (PT), activated partial thromboplastin time (aPTT), D-dimer, albumin, alanine aminotransferase (ALT), aspartate aminotransferase (AST), total bilirubin, serum potassium, sodium, creatinine, creatine kinase (CK), lactate dehydrogenase (LDH), u-Hydroxybutyrate dehydrogenase (HBDH), and C-reactive protein (CRP). In some embodiments, a method disclosed herein is used for adverse clinical outcome prediction of patients such as COVID-19 patients.

In some embodiments, a baseline model is used in performance comparisons. In some embodiments, a baseline model includes a diagnosis model purely based on medical images (denoted as Image-only). In some embodiments, a baseline model includes a traditional non-unified early fusion method with multimodal input data. In some embodiments, a baseline model includes a traditional non-unified late fusion method with multimodal input data. In some embodiments, a baseline model includes a transformer-based multimodal classification method. In some embodiments, a baseline model includes GIT and/or Perceiver.

In some embodiments, an image-only model is built on a transformer-based deep neural network for image understanding. In some embodiments, an image-only model is built on top of ViT, for example for pulmonary disease identification. In some embodiments, a model disclosed herein comprises a network architecture having 12 blocks and each block has a self-attention layer, a multi-layer perceptron (MLP), and two-layer normalization layers. In some embodiments, there are two fully-connected (FC) layers in each MLP, where the number of hidden nodes is 3,072. In some embodiments, the input size of the first FC layer is 768. In some embodiments, between the two FC layers, a GeLU activation function is inserted. In some embodiments, after each FC layer, a dropout layer is added, where the dropout rate is optionally set to 0.3. In some embodiments, the output size of the second FC layer is also 768. In some embodiments, each input image is divided into a number of 16×16 patches. In some embodiments, the output CLS token is used for performing the final classification. In some embodiments, the binary cross-entropy loss is used as the cost function during the training stage. In some embodiments, before the training stage, supervised ViT pre-training is performed on MIMIC-CXR to obtain visual representations with more generalization power. In some embodiments, in the task of rapid triage of COVID-19 patients, pneumonia lesions are first segmented from CT scans, then used to train multiple machine learning models (i.e., logistic regression, random forest, support vector machine, MLP, and LightGBM) using image features extracted from the segmented lesion areas, and finally the optimal model is chosen according to their performance on the validation set.

In some embodiments, a baseline model uses an archetypical non-unified approach to fuse multimodal input data for diagnosis. For better adaptation to different scenarios, different non-unified models can be adopted in different tasks. Specifically, an early fusion method is used, for example for pulmonary disease identification. In some embodiments, a ViT model extracts image features from radiographs, and the feature vector at its CLS token is taken as the representation of the input image. Similar to the image-only baseline, supervised pre-training on MIMIC-CXR can be applied to the ViT to obtain more powerful visual features before the formal task was carried out. To process the three types of clinical data (i.e., the chief complaint, demographics, and laboratory-test results), three independent MLPs are employed to convert different types of textual clinical data to features, which are then concatenated with the image representation. The rationale behind is that both images and textual data should be represented in the same feature space for the purpose of cross reference. Since the chief complaint includes unstructured texts, they first need to be transformed into structured items. To achieve this goal, an entity recognition model can be trained to highlight relevant clinical symptoms in the chief complaint. Next, BERT can be used to extract features for all such symptoms, to which average pooling is applied to produce a holistic representation for each patient's chief complaint. Then, a three-layer MLP can be used to further transform this holistic feature into a latent space similar to that of the image representation. In some embodiments, the input size of this three-layer MLP is 768, and the output size is 512. In some embodiments, the number of hidden nodes is 1,024. In some embodiments, after each FC layer, a ReLU activation and a dropout layer with the dropout rate set to 0.3 is added. In some embodiments, for laboratory-test results, an MLP is applied with the same architecture but independent weight parameters to transform those test results into a one-dimensional feature vector. In some embodiments, the input size of the laboratory-test MLP is 92 and the output size is 512. In some embodiments, the MLP model for demographics has two FC layers, where the input size is 2 and the output size is 512. In some embodiments, the hidden layer has 512 nodes. In some embodiments, the feature fusion module includes the concatenation operation and a three-layer MLP with the number of hidden nodes set to 1,024. The output from the MLP in the feature fusion module is passed to the final classification layer for making diagnostic decisions. During the training stage, the ViT-like model and all MLPs are joint-trained using the binary cross-entropy loss. As for the late fusion baseline, the predictions of the image- and text-based classifiers are ensembled. Specifically, a ViT model with radiographs and their associated labels are trained. To construct the input to the text-based classifier, laboratory-test results, demographics, and the holistic representation (obtained via averaging extracted features of symptoms, similar to the early fusion method) of the chief complaint are concatenated. The constructed input through a three-layer MLP is forwarded, whose input and output dimensions are 862 and 8, respectively. Then, the MLP with the same labels was used for training the ViT model. Finally, the predicted probabilities of the image- and text-based classifiers are averaged to obtain the final prediction.

In some embodiments, for adverse clinical outcome prediction for patients with COVID-19, an early fusion method is used, where image features, structured chief complaint (comorbidities and symptoms), and laboratory-test results have been concatenated as the input. In some embodiments, multiple machine learning models are trained and the optimal model is chosen using artificial rules. In some embodiments, for the late fusion baseline, five machine learning models (i.e., logistic regression, random forest, support vector machine, MLP, and LightGBM) are trained for image features, structured chief complaints, and laboratory-test results, respectively. Then, the average of the predicted probabilities of these fifteen machine learning models are taken as the adverse outcome prediction.

GIT 33 is a generative image-to-text transformer that unifies vision-language tasks. GIT-Base can be taken as a baseline in the comparisons. Its image encoder is a ViT-like transformer, and its text decoder consists of six standard transformer blocks²⁴. In some embodiments, the officially released pre-trained model can be fine-trained on custom datasets. In some embodiments, the same set of fine-tuning hyper-parameters used for the unified model disclosed herein can be adopted. In the pulmonary disease identification task, each radiograph is first forwarded through the image encoder to extract an image feature. Next, this image feature is concatenated with the averaged word embedding (using BERT) of the chief complaint as well as the feature vectors of the demographics and laboratory-test results. The concatenated features are then passed to the text decoder to make diagnostic predictions. In the task of adverse clinical outcome prediction of COVID-19 patients, the image features of CT slices are first averaged. Then, the averaged image feature is concatenated with the feature vectors of the clinical comorbidities and symptoms, laboratory-test results, and demographics. The concatenated multimodal features are forwarded through the text decoder to predict adverse outcomes of patients with COVID-19.

Perceiver is a transformer-based model³⁰ from DeepMind, proposed for tackling the classification problem with multimodal input data. There also exists a variant of Perceiver³⁰, Perceiver IO⁴³, which introduces the output query on top of Perceiver to handle additional types of tasks. As making diagnostic decisions can be considered as a type of classification, Perceiver instead of Perceiver IO can be adopted as one of the baseline models. The Perceiver architecture follows the setting for ImageNet classification^{44,30}, and has six cross-attention modules. Each cross-attention module is followed by a latent transformer with six self-attention blocks. The input of Perceiver consists of two arrays: the latent array and byte array. Following³⁰, the latent array is initialized using a truncated zero-mean normal distribution with standard deviation set to 0.02 and truncation bounds set to [−2, 2]. The byte array consists of multimodal data. In the pulmonary disease identification task, the input image is first flattened into a one-dimensional vector. Then, it is concatenated with the averaged word embedding (using BERT) of the chief complaint as well as one-dimensional feature vectors of the input demographics and laboratory-test results. This results in a long one-dimensional vector, which is taken as the byte array. In the task of adverse clinical outcome prediction of COVID-19, the input image is flattened into a one-dimensional vector, which is then concatenated with the feature vectors of the clinical comorbidities and symptoms, laboratory-test results, and demographics. The learning process of Perceiver can be summarized as follows: the latent array evolves by iteratively extracting higher-quality features from the input byte array by alternating cross-attention and latent self-attention computations. Finally, the transformed latent array serves as the representation used for diagnosis. Note that similar to the image-only and non-unified baselines, Perceiver can be pre-trained on MIMIC-CXR. During pre-training, zero padding can be used in the byte array for the non-existent clinical text in every multimodal input.

In some embodiments, a unified model disclosed herein comprises forwarding multimodal input data (e.g., medical images and textual clinical information) to a unified multimodal diagnostic transformer for acquiring prediction logits. In some embodiments, during the training stage, a binary cross-entropy loss is computed between the logits and ground-truth labels. In some embodiments, pulmonary disease annotations and/or real adverse clinical outcomes are used as the ground-truth labels. In some embodiments, pulmonary disease annotations are used as the ground-truth labels for identification of pulmonary disease. In some embodiments, real adverse clinical outcomes are used as the ground-truth labels for prediction of adverse clinical outcomes in patients with COVID-19.

In some embodiments, a unified transformer disclosed herein comprises two, three, four, five, or more starting layers. In some embodiments, the unified transformer comprises a starting layer for embedding the tokens from the input image, and a starting layer for embedding the tokens from the text data. In some embodiments, the unified transformer consists of a starting layer for embedding the tokens from the input image, and a starting layer for embedding the tokens from the text data. In some embodiments, the unified transformer comprises two, three, four, five, or more stacked bi-directional multimodal attention blocks. In some embodiments, the unified transformer comprises two stacked bi-directional multimodal attention blocks for learning fused mid-level representations by capturing interconnections among tokens from the same modality and across different modalities. In some embodiments, the unified transformer comprises two, three, four, five, six, seven, eight, nine, ten, or more stacked self-attention blocks, e.g., for learning holistic multimodal representations and enhancing their discriminative power. In some embodiments, the unified transformer comprises one or more classification heads each for producing prediction logits.

In some embodiments, the unified transformer comprises: (i) starting layers for embedding the tokens from the input image and text, (ii) stacked bidirectional multimodal attention blocks for learning fused mid-level representations by capturing interconnections among tokens from the same modality and across different modalities, (iii) stacked self-attention blocks for learning holistic multimodal representations and enhancing their discriminative power, and (iv) a classification head for producing prediction logits. In some embodiments, the unified transformer comprises: (i) two starting layers for embedding the tokens from the input image and text, respectively, (ii) stacked bidirectional multimodal attention blocks for learning fused mid-level representations by capturing interconnections among tokens from the same modality and across different modalities, (iii) ten stacked self-attention blocks for learning holistic multimodal representations and enhancing their discriminative power, and (iv) a classification head for producing prediction logits.

In some embodiments, the multimodal input data for the pulmonary disease identification task can comprise: a radiograph, a unstructured chief complaint that includes history of present and past illness, one or more laboratory-test results, each patient's gender, and age, which are denoted as x^{I}, x^{cc}, x^{lab}, x^{sex}, and x^{age}, respectively. In some embodiments, x^{I} is passed to a convolutional layer, which produces a sequence of visual tokens. In some embodiments, learnable 1D positional embedding^{21,2} and dropout can be added to every visual token to obtain a sequence of image patch tokens

$${X}_{1 : N}^{I} .$$

In some embodiments, word tokenization is applied to x^{cc} to encode each word from the unstructured chief complaint. In some embodiments, a pre-trained BERT²³ is used to generate an embedded feature vector for each word in x^{cc}, after which a sequence of word tokens

$${X}_{1 : {N}^{c c}}^{c c}$$

is obtained. In some embodiments, a similar tokenization procedure can be applied to x^{lab}, where min-max scaling is first employed to normalize every component of x^{lab}. Each normalized component is then passed to a shared linear projection layer to obtain a sequence of latent embeddings

$${X}_{1 : {N}^{l a b}}^{l a b} .$$

In some embodiments, linear projections on x^{sex} and x^{age} can be performed to obtain encoded feature vectors X^{sex} and X^{age}. Subsequently, in some embodiments,

$$\{ {X}_{1 : {N}^{cc}}^{cc} , {X}_{1 : {N}^{l a b}}^{l a b} , {X}^{sex} , {X}^{a g e} \}$$

can be concatenated together to produce a sequence of clinical text tokens

$${X}_{1 : \hat{N}}^{T} ,$$

where \hat{N}=N^{cc}+N^{lab}+2. In some embodiments, N^{cc} and N^{lab} can be set to 40 and 92, respectively.

In some embodiments, the multimodal input data for the adverse clinical outcome prediction of COVID-19 patients can comprise: a set of CT slices, structured chief complaint (comorbidities and symptoms), laboratory-test results, each patient's gender and age, which are denoted as x^{I}, x^{cc}, x^{lab}, x^{sex}, and x^{age}. In some embodiments, each CT slice is converted to a sequence of image patch tokens

$${X}_{1 : N}^{I}$$

as in the first task. In some embodiments, different from the first task, the chief complaint is structured. In some embodiments, to convert x^{cc} to tokens, a shared linear projection is conducted to each component, which generates a sequence of embeddings

$${X}_{1 : {N}^{c c}}^{c c} .$$

In some embodiments, a linear projection layer is applied to x^{lab} to acquire

$${X}_{1 : {N}^{l a b}}^{l a b} .$$

In some embodiments, linear projections is performed to obtain encoded X^{sex} and X^{age} as in the first task. In some embodiments,

$$\{ {X}_{1 : {N}^{cc}}^{cc} , {X}_{1 : {N}^{l a b}}^{l a b} , {X}^{sex} , {X}^{a g e} \}$$

is directly concatenated to produce \hat{N} clinical text tokens

$${X}_{1 : \hat{N}}^{T} ,$$

where \hat{N}=N^{cc}+N^{lab}+2. In some embodiments, N^{cc} and N^{lab} are set to 16 and 19, respectively.

In some embodiments, the first two layers of MDT are two stacked bi-directional multimodal attention blocks. In some embodiments, suppose the input of the first bi-directional multimodal attention block consists of

$${X}_{I}^{l} and {X}_{T}^{l} ,$$

where l (=0) stands for the layer index,

$${X}_{I}^{0} = {X}_{1 : N}^{I}$$

denotes the assembly of image patch tokens, and

$${X}_{T}^{0} = {X}_{1 : \hat{N}}^{T}$$

represents the bag of clinical text tokens. In some embodiments, the process of generating the query, key, and value matrices for each modality in the bi-directional multimodal attention block is as follows:

$${Q}_{I}^{l} , {K}_{I}^{l} , {V}_{I}^{l} = LP ( Norm ( {X}_{I}^{l} ) ) ,$$

$${Q}_{T}^{l} , {K}_{T}^{l} , {V}_{T}^{l} = LP ( Norm ( {X}_{T}^{l} ) ) ,$$

where LP(·) and Norm(·) represent linear projection and layer normalization, respectively. In some embodiments, the forward pass inside a bi-directional multimodal attention block can be summarized as:

$${I}_{l}^{} = Attention ( {Q}_{I}^{l} , {K}_{I}^{l} , {V}_{I}^{l} ) + λ Attention ( {Q}_{I}^{l} , {K}_{T}^{l} , {V}_{T}^{l} ) ,$$

$${T}_{l}^{} = Attention ( {Q}_{T}^{l} , {K}_{T}^{l} , {V}_{T}^{l} ) + λ Attention ( {Q}_{T}^{l} , {K}_{I}^{l} , {V}_{I}^{l} ) ,$$

### Attention

$$( {Q}_{I}^{l} , {K}_{I}^{l} , {V}_{I}^{l} )$$

### and Attention

$$( {Q}_{T}^{l} , {K}_{T}^{l} , {V}_{T}^{l} )$$

capture the intra-modal connections in the image and text modalities, respectively. Attention

$$( {Q}_{I}^{l} , {K}_{T}^{l} , {V}_{T}^{l} )$$

### and Attention

$$( {Q}_{T}^{l} , {K}_{I}^{l} , {V}_{I}^{l} )$$

dig out the inter-modal connections between the image and text. Next, both intra- and inter-modal connections are encoded into latent representations [symbol] and [symbol] In some embodiments, Λ is set to 1.0. In some embodiments, Attention(Q, K, V) includes two matrix multiplications and one scaled softmax operation:

$$Attention ( Q , K , V ) = softmax ( \frac{{QK}^{⊤}}{\sqrt{{d}_{k}}} V ) ,$$

where τ stands for the matrix transpose operator, dₖ is a scaling hyper-parameter, which can be set to 64. In some embodiments, residual learning is introduced and the resulting [symbol] are forwarded to the following normalization layer and MLP:

$${X}_{I}^{l + 1} = MLP ( Norm ( {I}_{l}^{} ) ) + {\text{+X}}_{I}^{l} ,$$

$${X}_{T}^{l + 1} = MLP ( Norm ( {T}_{l}^{} ) ) + {\text{+X}}_{T}^{l} ,$$

$${X}_{I}^{l + 1} and {X}_{T}^{l + 1}$$

are passed to the next bi-directional multimodal attention block as the input, resulting in

$${X}_{I}^{l + 2} and {X}_{T}^{l + 2} .$$

In some embodiments, tokens in

$${X}_{I}^{l + 2} and {X}_{T}^{l + 2}$$

are combined to produce a bag of unified tokens, which are passed to the following self-attention blocks. In some embodiments, multiple heads can be allocated in both bi-directional multimodal attention and self-attention blocks. In some embodiments, the number of heads is set to 12. In some embodiments, the multi-head mechanism allows the model to perform attention operations in multiple representation subspaces simultaneously and aggregate the results afterwards.

In some embodiments, average pooling is applied to the unified tokens generated from the last self-attention block to obtain a holistic multimodal representation for medical diagnosis. This representation is passed to a two-layer MLP to produce final prediction logits. In some embodiments, during the training stage, the binary cross-entropy loss is calculated between these logits and their corresponding pulmonary disease annotations (the first task) or real adverse clinical outcomes (the second task). In some embodiments, a loss function value is computed for every patient case. In some embodiments, in the first task, each patient case contains one radiograph and related textual clinical information. In some embodiments, in the second task, each patient case involves multiple CT slices, and these CT slices share the same textual clinical information. In some embodiments, each CT slice is forwarded and its accompanying textual clinical information to MDT to obtain one holistic representation. In some embodiments, multiple CT slices are used, and a number of holistic representations (equal to the number of CT slices) are obtained for the same patient. In some embodiments, an average pooling over these holistic representations is performed to compute an averaged representation, which is finally passed to a two-layer MLP and the binary cross-entropy loss.

Exemplary embodiments provided herein include:

Embodiment 1: A method for training a model for providing a medical diagnosis, wherein the model comprises a free-form embedding layer, an image embedding layer, a bidirectional multimodal attention block, a self-attention block, and a classification head, the method comprising: (a) obtaining multimodal input data of a subject, wherein the multimodal input data comprise a set of clinical text data and a set of medical images; (b) tokenizing and embedding the set of clinical text data in the free-form embedding layer to generate a sequence of clinical text tokens, and tokenizing and embedding the set of medical images in the image embedding layer to generate a sequence of image patch tokens; (c) passing the sequence of clinical text tokens and the sequence of image patch tokens to the bidirectional multimodal attention block to generate a bag of unified tokens, wherein the bidirectional multimodal attention block applies (i) a first attention to capture intramodal connections in the set of clinical text data, (ii) a second attention to capture intramodal connections in the set of medical images, and (iii) a third attention to capture intermodal connections between the set of clinical text data and the set of medical images, and wherein both the intramodal connections and the intermodal connections are encoded into latent representations, and representations across modalities are learned and fused simultaneously; and (d) passing the bag of unified tokens to the self-attention block to learn a holistic multimodal representation of medical diagnosis for inputting to the classification head, thereby training the model.

Embodiment 2: The method of Embodiment 1, wherein the free-form embedding layer is configured to convert unstructured and structured texts into uniform text tokens.

Embodiment 3: The method of Embodiment 1 or Embodiment 2, wherein the free-form embedding layer is configured to convert a chief complaint, a laboratory test result, and/or a demographic information into clinical text tokens.

Embodiment 4: The method of any one of Embodiments 1-3, wherein the free-form embedding layer comprises a dropout layer for clinical text tokens.

Embodiment 5: The method of any one of Embodiments 1-4, wherein the image embedding layer comprises a convolutional layer configured to produce a sequence of visual tokens.

Embodiment 6: The method of any one of Embodiments 1-5, wherein the image embedding layer comprises a dropout layer for image patch tokens.

Embodiment 7: The method of any one of Embodiments 1-6, wherein the model comprises two or more stacked bidirectional multimodal attention blocks.

Embodiment 8: The method of any one of Embodiments 1-7, wherein each bidirectional multimodal attention block comprises two-layer normalization layers.

Embodiment 9: The method of any one of Embodiments 1-8, wherein the bidirectional multimodal attention block comprises a bidirectional multimodal attention layer.

Embodiment 10: The method of any one of Embodiments 1-9, wherein the bidirectional multimodal attention block comprises a multilayer perceptron.

Embodiment 11: The method of any one of Embodiments 1-10, wherein the model comprises two or more stacked self-attention blocks.

Embodiment 12: The method of any one of Embodiments 1-11, wherein the model comprises ten self-attention blocks.

Embodiment 13: The method of any one of Embodiments 1-12, wherein each self-attention block comprises two-layer normalization layers.

Embodiment 14: The method of any one of Embodiments 1-13, wherein each self-attention block comprises a self-attention layer.

Embodiment 15: The method of any one of Embodiments 1-14, wherein each self-attention block comprises a multilayer perceptron.

Embodiment 16: The method of any one of Embodiments 1-15, wherein the classification head is configured to identify a disease or condition in a patient.

Embodiment 17: The method of any one of Embodiments 1-16, wherein the classification head is configured to predict an adverse clinical outcome in a patient.

Embodiment 18: The method of any one of Embodiments 1-17, wherein the set of clinical text data comprises a chief complaint, a demographic information, and a laboratory test report.

Embodiment 19: The method of Embodiment 18, wherein the chief complaint comprises unstructured data.

Embodiment 20: The method of Embodiment 18 or Embodiment 19, wherein the chief complaint comprises structured data.

Embodiment 21: The method of any one of Embodiments 18-20, wherein the set of clinical text data comprises: an unstructured chief complaint comprising a history of present and past illness; a comorbidity; a symptom; gender; and/or age.

Embodiment 22: The method of any one of Embodiments 1-21, wherein the set of medical images comprises one or more CT images, one or more X-ray images, one or more optical coherence tomography (OCT) images, one or more retinal fundus photographs, one or more fundus fluorescein angiography (FFA) images, one or more indocyanine green angiography (ICGA) images, or a combination thereof.

Embodiment 23: The method of any one of Embodiments 1-22, wherein in (b), encoded feature vectors for each category of data in the set of clinical text data are concatenated to produce the sequence of clinical text tokens.

Embodiment 24: The method of any one of Embodiments 1-23, wherein in (c), the bidirectional multimodal attention block comprises multiple heads configured to perform attention operations in multiple representation subspaces simultaneously and aggregate results from the multiple representation subspaces afterwards.

Embodiment 25: The method of any one of Embodiments 1-24, wherein in (d), the self-attention block comprises multiple heads configured to perform attention operations in multiple representation subspaces simultaneously and aggregate results from the multiple representation subspaces afterwards.

Embodiment 26: The method of any one of Embodiments 1-25, comprising applying average pooling to the bag of unified tokens generated from the self-attention block.

Embodiment 27: A method for training a model for providing a medical diagnosis, wherein the model comprises a first embedding layer, a second embedding layer that differs from the first embedding layer, a bidirectional multimodal attention block, a self-attention block, and a classification head, the method comprising: (a) obtaining multimodal input data of a subject, wherein the multimodal input data comprise data in a first modality and data in a second modality which differs from the first modality; (b) tokenizing and embedding the data in the first modality in the first embedding layer to generate a sequence of first modality tokens, and tokenizing and embedding the data in the second modality in the second embedding layer to generate a sequence of second modality tokens; (c) passing the sequence of first modality tokens and the sequence of second modality tokens to the bidirectional multimodal attention block to generate a bag of unified tokens, wherein the bidirectional multimodal attention block applies (i) a first attention to capture intramodal connections in the data in the first modality, (ii) a second attention to capture intramodal connections in the data in the second modality, and (iii) a third attention to capture intermodal connections between the data in the first modality and the data in the second modality, and wherein both the intramodal connections and the intermodal connections are encoded into latent representations, and representations across modalities are learned and fused simultaneously; and (d) passing the bag of unified tokens to the self-attention block to learn a holistic multimodal representation of medical diagnosis for inputting to the classification head, thereby training the model.

Embodiment 28: The method of Embodiment 27, wherein the first modality is text data.

Embodiment 29: The method of Embodiment 27 or Embodiment 28, wherein the data in the first modality comprise unstructured data.

Embodiment 30: The method of any one of Embodiments 27-29, wherein the second modality is image data.

Embodiment 31: A method of generating a medical diagnosis for a subject, the method comprising: receiving a prompt for obtaining the medical diagnosis and a set of data related to the subject, and generating the medical diagnosis by inputting the prompt and the set of data in a trained model generated by the method of any one of Embodiments 1-30.

Embodiment 32: The method of Embodiment 31, wherein the medical diagnosis is identification of a pulmonary disease or condition in the subject.

Embodiment 33: The method of Embodiment 31 or Embodiment 32, wherein the medical diagnosis is prediction of an adverse clinical outcome in the subject.

Embodiment 34: A system comprising: at least one hardware processor; and one or more software modules configured to, when executed by the at least one hardware processor, perform the method of any one of Embodiments 1-33.

Embodiment 35: A non-transitory computer-readable medium having instructions stored thereon, wherein the instructions, when executed by a processor, cause the processor to perform the method of any one of Embodiments 1-33.

Embodiment 36: A system comprising: at least one hardware processor; non-transitory computer-readable medium coupled to at least one hardware processor, optionally wherein the coupling is over a network; and instructions stored in the non-transitory computer-readable medium, wherein the instructions when implemented by the processor, configure the system to perform the method of any one of Embodiments 1-33.

### EXAMPLES

The following examples are included for illustrative purposes only and are not intended to limit the scope of the present disclosure.

A transformer-based representation-learning model that processes multimodal input in a unified manner outperformed non-unified multimodal models in two clinical-diagnostic tasks. Rather than learning modality-specific features, the model used embedding layers to convert images and unstructured and structured text into visual tokens and text tokens, and bidirectional blocks with intramodal and intermodal attention to learn holistic representations of radiographs, the unstructured chief complaint and clinical history, structured clinical information such as laboratory-test results and patient demographic information. The unified model outperformed an image-only model and non-unified multimodal diagnosis models in the identification of pulmonary disease (by 12% and 9%, respectively) and in the prediction of adverse clinical outcomes in patients with COVID-19 (by 29% and 7%, respectively). Leveraging unified multimodal transformer-based models may help streamline the triaging of patients and facilitate the clinical decision process.

### Example 1—Dataset Characteristics for Multimodal Diagnosis

The first dataset focused on pulmonary diseases. Consecutive chest X-rays were retrospectively collected from 51,511 patients between Nov. 27, 2008, and May 31, 2019, at West China Hospital, which is the largest tertiary medical center in western China covering a 100 million population. Each patient is associated with at least one radiograph, a short piece of unstructured chief complaint, history of present and past illness, demographics, and a complete laboratory-test report. The dataset is built for eight pulmonary diseases, including chronic obstructive pulmonary disease (COPD), bronchiectasis, pneumothorax, pneumonia, interstitial lung disease (ILD), tuberculosis, lung cancer, and pleural effusion. Discharge diagnoses are extracted from discharge summary reports following the standard process described in previous study¹⁶, and taken as the ground-truth disease labels. The discharge summary reports were produced as follows. An initial report was written by a junior physician, which was then reviewed and confirmed by a senior physician. In case of any disagreement, the final decision was made by a departmental committee comprised of at least three senior physicians.

The built dataset consisted of 72,283 data samples, among which 40,126 samples are normal. The distribution of diseases (i.e., the number of relevant cases) is as follows: COPD (4,912), bronchiectasis (676), pneumothorax (2,538), pneumonia (21,409), ILD (3,283), tuberculosis (938), lung cancer (2,651) and pleural effusion (4,713). The performance metric is the area under the receiver operating characteristic curve (AUROC). This dataset was split into training, validation, and testing sets according to each patient's admission date. Specifically, the training set included 44,628 patients admitted between Nov. 27, 2008, and Jun. 1, 2018. And the validation set included 3,325 patients admitted between Jun. 2, 2018 and Dec. 1, 2018. Finally, the trained and validated unified system was tested on 3,558 patients admitted between Dec. 2, 2018 and May 31, 2019. Although this was a retrospective study, the data splitting scheme followed the practice of a prospective study, thus creating a more challenging and realistic setting to verify the effectiveness of different multimodal medical diagnosis systems, in comparison to data splitting schemes based on random sampling.

The second dataset MMC (i.e., multimodal COVID-19 dataset)¹⁹, on which the unified model was trained and evaluated, consisting of chest CT images and structured clinical information (e.g., chief complaint that comprises comorbidities and symptoms, demographics, laboratory-test results, etc) collected from COVID-19 patients. The CT images were associated with inpatients with laboratory-confirmed COVID-19 infection between Dec. 27, 2019 and Mar. 31, 2020. There were three types of adverse events that could happen to patients in MMC, namely admission to ICU, mechanical ventilation (MV), and death. The training and validation sets came from 17 hospitals, and the training set had 1,164 labeled cases (70%) while the validation set had 498 labeled ones (30%). Next, the trained model with the best performance on the validation set was chosen and tested on the independent testing set, which is comprised of 700 cases collected from 9 external medical centers. The distribution of the three events in the testing set was as follows: ICU (155), MV (94), Death (59). This was an imbalanced classification problem where the majority of patients did not have any adverse outcomes. Against this background, the area under the precision-recall curve (AUPRC) was used instead of AUROC as the performance metric, which focused more on identifying adverse events (i.e., ICU, MV, and Death).

For the pulmonary disease identification task, each radiograph was resized to 256×256 pixels during the training stage, then crop a random portion of each image, where the area ratio between the cropped patch and the original radiograph is randomly determined between 0.09 and 1.0. The cropped patch is resized to 224×224, after which a random horizontal flip is applied to increase the diversity of training data. In the validation and testing stages, each radiograph is first resized to 256×256 pixels, and then a square patch at the image center is cropped. The size of the square crop is 224×224. The processed radiographs are finally passed to the Image-only model, Non-unified-Chest, Perceiver, and the unified model as input images. In the task of adverse clinical outcome prediction of COVID-19 patients, the input images are CT scans. The lesion detection and segmentation methodologies proposed in⁴⁶ were used. This is a deep learning algorithm based on a multi-view feature pyramid convolutional neural network^{47,48}, which performs lesion detection, segmentation, and localization. This neural network was trained and validated on 14,435 participants with chest CT images and definite pathogen diagnosis. On a per-patient basis, the algorithm showed superior sensitivity of 1.00 [95% CI. 0.95, 1.00] and an F1-score of 0.97 in detecting lesions from CT images of COVID-19 pneumonia patients. Adverse clinical outcomes of COVID-19 are presumed to be closely related to the characteristics of pneumonia lesion areas. For each patient's case, a 3D CT subvolume was cropped by computing the minimum 3D bounding box enclosing all pneumonia lesions. Next, all 3D subvolumes were resized from different patients to a uniform size, which is 224×224×64. At the end, 16 evenly spaced slices were sampled from every 3D subvolume along its third dimension.

Before the formal training procedure was performed, the MDT was pre-trained on MIMIC-CXR⁴², similar to the case of the baseline models. Similar to Perceiver, during pre-training, zero padding for non-existent textual clinical information was used in every multimodal input. In the formal training stage, AdamW⁴⁹ was used as the default optimizer as it was found empirically to give rise to better performance on baseline models and the unified model. The initial learning rate is set to 3e-5 and the weight decay is 1e-2. Each model was trained for 30 epochs and decrease the initial learning rate by a factor of 10 at the 20-th epoch. The batch size is set to 256 in the training stage of both tasks. It is worth noting that in the task of adverse clinical outcome prediction of COVID-19, holistic feature representations were first extracted from 16 CT slices (cropped and sampled from the same CT volume). Next, average pooling was applied to these 16 holistic features to obtain an averaged representation, which represents all pneumonia lesion areas in the entire CT volume. The binary cross-entropy loss is then computed on top of this averaged representation. During the training stage, the model performance was evaluated on the validation set and calculate the validation loss after each epoch. The model checkpoint that produces the lowest validation loss is saved and then tested on the testing set. Learnable positional embeddings were employed in all ViT models. The unified model is implemented using PyTorch⁵⁰ and the training stage is accelerated using NVIDIA Apex with the mixed-precision strategy⁵¹. In practice, the training stage of either task was finished within one day using four NVIDIA GPUs.

The standard attention analysis strategy was adopted for vision transformers. For each layer in the transformer, the attention weights were averaged across multiple heads (as multi-head self-attention was used in the unified model) to obtain an attention matrix. To account for residual connections, an identity matrix was added to each attention matrix and normalize the resulting weight matrices. Next, the weight matrices were recursively multipled from different layers of the transformer. Finally, an attention map was obtained that includes the similarity between every input token and the CLS token. Since the CLS token is used for diagnostic predictions, these similarities indicate the relevance between the input tokens and prediction results, which can be used for visualization. For cross-attention results, visualization with Grad-CAM⁵² was performed.

Non-parametric bootstrap sampling is used to calculate 95% confidence intervals. Specifically, 1,000 bootstrap samples were repeatedly drawn from the unseen test set. Each bootstrap sample is obtained through random sampling with replacement, and its size is the same as the size of the test set. AUROC (the first task) or AUPRC (the second task) was then computed on each bootstrap sample, after which 1,000 AUROC or AUPRC values were had. Finally, these performance results were sorted and report the values at 2.5 and 97.5 percentiles, respectively.

To demonstrate the statistical significance of the experimental results, the experiments of the unified model and the best performing baseline (i.e., Perceiver) were first repeated five times with different random seeds. Then, P-values between the mean performance of the unified model and the best baseline results were calculated using the independent two-sample t-test (two-sided).

Code is available at github.com/RL4M/IRENE.

### Example 2—Pulmonary Disease Identification

The effectiveness of the unified model is validated on pulmonary disease identification. The unified model outperformed previous image-only and non-unified diagnostic counterparts by approximately 12% and 9% (cf. FIG. 1C), respectively. When compared to human experts (cf. FIG. 1E) in pulmonary disease identification, the unified model clearly surpassed junior physicians (with <7 years of experience) in the diagnosis of all eight diseases, and delivered a performance comparable to or better than that of senior physicians (with more than 7 years of experience) on six diseases.

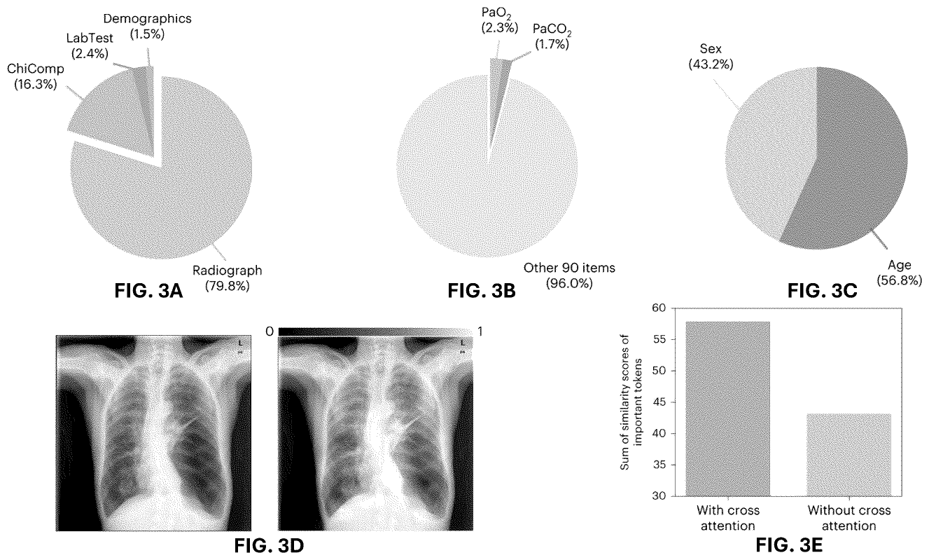

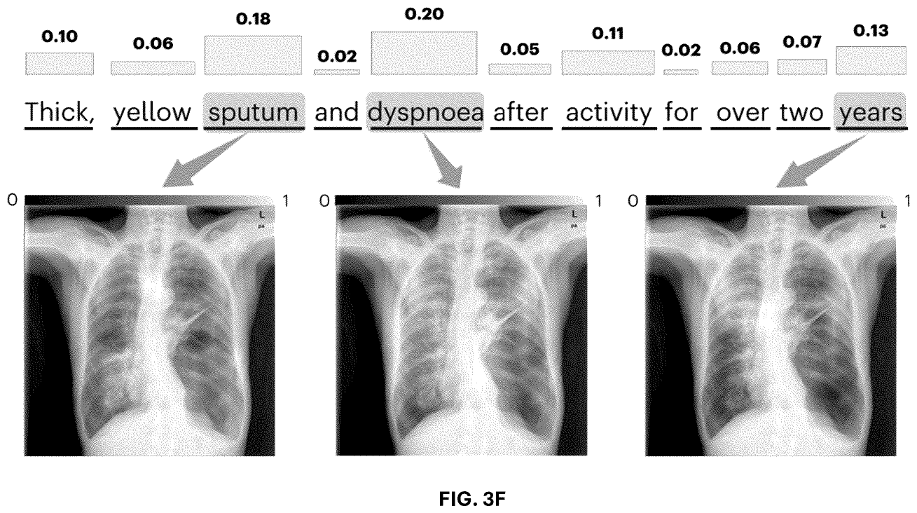

Table 1 and FIGS. 3A-3F present the experimental results from the unified model and other methods on the dataset for pulmonary disease identification.

TABLE 1
Comparison with baseline models in the task of pulmonary disease identification. The baseline
models include the image-only model, the early fusion method, the late fusion approach,
and two recent transformer-based multimodal classification models (i.e., GIT and Perceiver).
95% CI denotes the 95% confidence interval. The evaluation metric is AUROC.

| | | | | | | | | Lung | Pleural |
|---|---|---|---|---|---|---|---|---|---|
| Method | Mean | COPD | Bronchiectasis | Pneumothorax | Pneumonia | ILD | Tuberculosis | cancer | effusion |
| Image- | 0.805 | 0.847 | 0.746 | 0.789 | 0.845 | 0.799 | 0.769 | 0.825 | 0.819 |
| only | (0.802, | (0.845, | (0.743, | (0.786, | (0.843, | (0.796, | (0.765, | (0.821, | (0.817, |
| | 0.808) | 0.851) | 0.748) | 0.791) | 0.848) | 0.801) | 0.772) | 0.830) | 0.822) |
| Early | 0.835 | 0.895 | 0.772 | 0.810 | 0.873 | 0.824 | 0.793 | 0.871 | 0.842 |
| Fusion | (0.832, | (0.893, | (0.768, | (0.807, | (0.870, | (0.822, | (0.791, | (0.868, | (0.839, |
| | 0.839) | 0.898) | 0.775) | 0.812) | 0.875) | 0.827) | 0.796) | 0.875) | 0.845) |
| Late | 0.826 | 0.888 | 0.765 | 0.822 | 0.870 | 0.804 | 0.770 | 0.839 | 0.850 |
| Fusion | (0.823, | (0.885, | (0.763, | (0.820, | (0.868, | (0.802, | (0.767, | (0.836, | (0.847, |
| | 0.828) | 0.890) | 0.767) | 0.825) | 0.872) | 0.805) | 0.772) | 0.841) | 0.852) |
| GIT | 0.848 | 0.911 | 0.798 | 0.824 | 0.895 | 0.819 | 0.807 | 0.872 | 0.858 |
| | (0.844, | (0.907, | (0.796, | (0.821, | (0.893, | (0.816, | (0.804, | (0.871, | (0.855, |
| | 0.850) | 0.913) | 0.800) | 0.827) | 0.898) | 0.821) | 0.810) | 0.873) | 0.860) |
| Perceiver | 0.858 | 0.910 | 0.788 | 0.846 | 0.903 | 0.830 | 0.825 | 0.890 | 0.872 |
| | (0.855, | (0.907, | (0.784, | (0.842, | (0.901, | (0.827, | (0.823, | (0.887, | (0.869, |
| | 0.861) | 0.912) | 0.791) | 0.850) | 0.906) | 0.833) | 0.828) | 0.892) | 0.874) |
| Unified | 0.924 | 0.922 | 0.907 | 0.954 | 0.921 | 0.934 | 0.918 | 0.914 | 0.924 |
| Model | (0.921, | (0.920, | (0.903, | (0.952, | (0.918, | (0.929, | (0.917, | (0.911, | (0.921, |
| | 0.926) | 0.925) | 0.910) | 0.957) | 0.923) | 0.937) | 0.921) | 0.917) | 0.926) |

As shown in Table 1, the unified model significantly outperformed the image-only model, traditional non-unified early¹⁹ and late fusion²³ methods, and two recent transformer-based multimodal methods (i.e., Perceiver³⁰ and GIT³³) in identifying pulmonary diseases. The unified model achieved the highest mean AUROC (0.924, [95% CI: 0.921, 0.927]), about 12% higher than the image-only model (0.805, [95% CI: 0.802, 0.808]) that only takes radiographs as the input. In comparison to diagnostic decisions made by non-unified early fusion (0.835, [95% CI: 0.832, 0.839]) and late fusion (0.826, [95% CI: 0.823, 0.828]) methods, the unified model maintained an advantage of 9% at least. Comparing the unified model to GIT (0.848, [95% CI: 0.844, 0.850]), an advantage of over 7% was observed. Even when compared to Perceiver, the transformer-based multimodal classification model developed by DeepMind, the unified model still delivered competitive results, surpassing Perceiver (0.858 [95% CI: 0.855, 0.861]) by over 6%. When carefully checking each disease and comparing the unified model against the previous best result among all five baselines, among all eight pulmonary diseases, the unified model achieved the largest improvements on bronchiectasis (12%), pneumothorax (10%), ILD (10%), and tuberculosis (9%).

The unified model was compared against human experts, who were divided into two groups, one group of two junior physicians (with <7 years of experience) and the second group of two senior physicians (with >7 years of experience). For better comparison, the average performance within each group is presented in FIG. 1E. Specifically, annotations by human experts were extracted from electronic discharge diagnosis records. Notably, all physicians from the reader study did not participate in data annotation. The unified model exhibited advantages over the junior group on all eight pulmonary diseases, especially in the diagnosis of bronchiectasis (Junior, [FPR: 0.29, TPR: 0.58]), pneumonia (Junior, [FPR: 0.37, TPR: 0.76]), ILD (Junior, [FPR: 0.09, TPR: 0.63]), and pleural effusion (Junior, [FPR: 0.35, TPR: 0.86]), where FPR and TPR stood for the false and true positive rates, respectively. Compared to the senior group, the unified model was advantageous in the diagnosis of pneumonia (Senior, [FPR: 0.21, TPR: 0.80]), tuberculosis (Senior, [FPR: 0.07, TPR: 0.17]), and pleural effusion (Senior, [FPR: 0.25, TPR: 0.77]). In addition, the unified model performed comparably with senior physicians on COPD (Senior, [FPR: 0.07, TPR: 0.76]), ILD (Senior, [FPR: 0.09, TPR: 0.71]), and pneumothorax (Senior, [FPR: 0.08, TPR: 0.79]) while showing slightly worse performance on bronchiectasis (Senior, [FPR: 0.12, TPR: 0.82]) and lung cancer (Senior, [FPR: 0.08, TPR: 0.73]).

### Example 3—Adverse Clinical Outcome Prediction of COVID-19 Patients

The effectiveness of the unified model is validated on adverse clinical outcome prediction of COVID-19 patients. The unified model is employed to predict adverse clinical events of COVID-19 patients, i.e., admission to the intensive care unit (ICU), mechanical ventilation (MV) therapy, and death. Different from pulmonary disease identification, adverse clinical outcome prediction of COVID-19 patients relies more on textual clinical information. In this scenario, the unified model significantly outperforms non-unified approaches by over 7% (cf. FIG. 1D). Particularly noteworthy is the nearly 10-percent improvement that the unified model achieves on death prediction, demonstrating the potential in assisting doctors to take immediate steps for saving COVID-19 patients.

Triage of COVID-19 patients heavily depends on joint interpretation of chest CT scans and other non-imaging clinical information. In this scenario, the unified model exhibited even more advantages than it did in the pulmonary disease identification task. As shown in Table 2, the unified model consistently achieved impressive performance improvements on the prediction of the three adverse clinical outcomes of COVID-19 patients, i.e., admission to ICU, mechanical ventilation, and death.

TABLE 2
Comparison with baseline models in the task of adverse clinical outcome prediction
of COVID-19 patients. Five models are included in the comparison, which are the
image-only model, the early fusion method, the late fusion approach, and two recent
transformer-based multimodal classification models (i.e., GIT and Perceiver). 95%
CI denotes the 95% confidence interval. The evaluation metric is AUPRC.

| Method | Mean | Admission to ICU | Need for MV | Death |
|---|---|---|---|---|
| Image-only | 0.307 (0.237, 0.391) | 0.482 (0.355, 0.636) | 0.247 (0.136, 0.398) | 0.192 (0.073, 0.333) |
| Early Fusion | 0.521 (0.435, 0.614) | 0.665 (0.548, 0.774) | 0.551 (0.397, 0.699) | 0.346 (0.174, 0.544) |
| Late Fusion | 0.503 (0.422, 0.598) | 0.647 (0.535, 0.759) | 0.533 (0.388, 0.685) | 0.330 (0.164, 0.531) |
| GIT | 0.514 (0.442, 0.605) | 0.653 (0.546, 0.743) | 0.554 (0.411, 0.702) | 0.335 (0.168, 0.554) |
| Perceiver | 0.526 (0.448, 0.611) | 0.652 (0.529, 0.771) | 0.566 (0.406, 0.715) | 0.360 (0.201, 0.543) |
| The Unified | 0.592 (0.500, 0.682) | 0.712 (0.587, 0.834) | 0.624 (0.473, 0.754) | 0.441 (0.270, 0.617) |
| Model | | | | |

In terms of mean AUPRC, the unified model (0.592, [95% CI: 0.500, 0.682]) outperformed the image-only model (0.307, [95% CI: 0.237, 0.391]), early fusion model²² (0.521, [95% CI: 0.435, 0.614]), and late fusion model²³ (0.503, [95%: 0.422, 0.598]) by nearly 29%, 7%, and 9%, respectively. As for specific clinical outcomes, the unified model (0.712, [95% CI: 0.587, 0.834]) achieved about 5-percent AUPRC gain over the non-unified early fusion method (0.665, [95% CI: 0.548, 0.774]) in the prediction of admission to ICU. Similarly, in the prediction of MV, the unified model achieved an over 6-percent performance improvement when compared to the early fusion model. Last but not the least, the unified model (0.441, [95% CI: 0.270, 0.617]) was much more capable of predicting death than the image-only model (0.192, [95% CI: 0.073, 0.333]), early fusion model (0.346, [95%: 0.174, 0.544]), and late fusion model (0.335, [95% CI: 0.168, 0.554]). Compared to two transformer-based multimodal models, i.e., GIT and Perceiver, an advantage of over 6% on average was observed.

### Example 4—Impact of Different Modules and Modalities in the Unified Model

To investigate the impact of different modules and modalities, thorough ablative experiments were conducted and reported their results in Table 3.

TABLE 3
An ablation study of the unified model by removing or replacing individual components. HA (N)
denotes the presence of N bi-directional multimodal attention block(s) in the multimodal diagnosis
transformer (MDT) while the remaining blocks are self-attention blocks (twelve blocks in total).
Image denotes the input radiograph. Uni-direction means only compute text-to-image attention
in multimodal attention blocks were computed. ChiComp stands for the chief complaint. LabTest
denotes laboratory-test results. Tokenization stands for the tokenization procedures for the
chief complaint and laboratory-test results. The evaluation metric is AUROC.

| | HA | HA | HA | Uni- | | | | | |
|---|---|---|---|---|---|---|---|---|---|
| Row | (2) | (0) | (6) | direction | Image | ChiComp | LabTest | Tokenization | Mean |
| 0 | ✓ | | | | | ✓ | ✓ | ✓ | 0.924 (0.921, 0.926) |
| 1 | | ✓ | | | ✓ | ✓ | ✓ | ✓ | 0.858 (0.850, 0.867) |
| 2 | | | ✓ | | ✓ | ✓ | ✓ | ✓ | 0.905 (0.899, 0.910) |
| 3 | ✓ | | | ✓ | ✓ | ✓ | ✓ | ✓ | 0.884 (0.880, 0.888) |
| 4 | ✓ | | | | ✓ | | ✓ | ✓ | 0.860 (0.855, 0.864) |
| 5 | ✓ | | | | ✓ | ✓ | | ✓ | 0.882 (0.873, 0.891) |
| 6 | ✓ | | | | ✓ | ✓ | ✓ | | 0.894 (0.886, 0.900) |
| 7 | ✓ | | | | | ✓ | ✓ | ✓ | 0.543 (0.525, 0.569) |

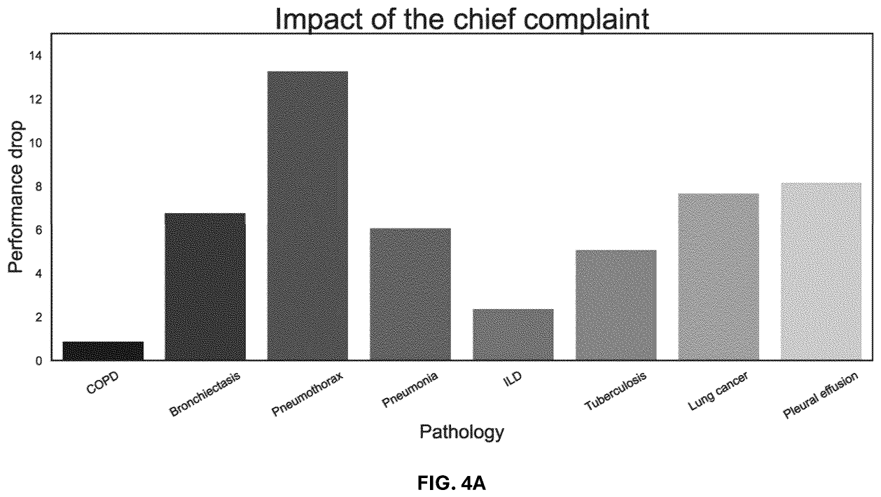

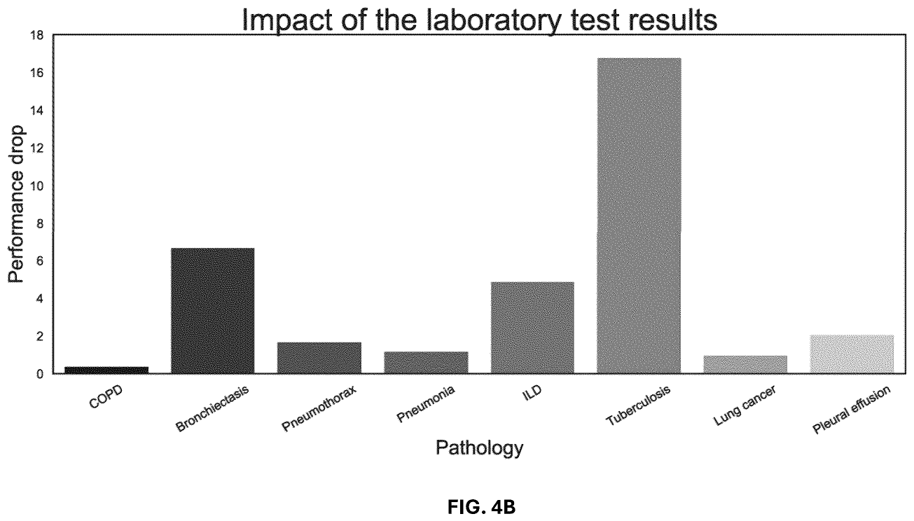

The impact of bi-directional multimodal attention blocks (rows 0-2) were investigated. Replacing all bi-directional multimodal attention blocks with self-attention blocks led to about 7-percent performance drop (from 0.924 to 0.858) in pulmonary disease identification. This phenomenon verified that directly learning progressively fused representations from raw data would deteriorate the diagnosis performance. On the contrary, simply increasing the number of bi-directional multimodal attention blocks from two to six did not bring obvious performance improvements (from 0.924 to 0.905), indicating that using two successive bi-directional multimodal attention blocks could be an optimal choice in the unified model. In row 3, the result of using uni-directional attention (i.e., text-to-image attention) is presented. Comparing row 0 with row 3, it is observed that bi-directional design brought a 4-percent performance gain (from 0.884 to 0.924). Next, the impact of clinical texts (rows 4 and 5) was studied. The first observation was that utilizing the complementary narrative chief complaint substantially boosted the diagnostic performance because removing chief complaint from the input data reduced model performance by 6% (from 0.924 to 0.860). Apart from chief complaint, the impact of laboratory-test results (row 5) was studied. Including laboratory-test results brings about a 4-percent performance gain (from 0.882 to 0.924). Then, the impact of tokenization procedures was investigated. Modelling the chief complaint and laboratory-test results of a patient as a sequence of tokens (row 0) did perform better than directly passing an averaged representation (row 6) to the model. This improvement brought by the tokenization of chief complaint and laboratory-test results verified the advantage of token-level intra- and inter-modal bi-directional multimodal attention, which exploited local interconnections among the word tokens of the clinical text and the image patch tokens of the radiograph in the input data. In the end, the impact of the input image in the unified model (row 7) resulted in a dramatic performance drop (from 0.924 to 0.543). This phenomenon indicated the vital role of the input radiograph in pulmonary disease identification. The impact of chief complaints (FIG. 4A) and laboratory-test results (FIG. 4B) on each respiratory disease were then investigated. When either chief complaints or the laboratory-test results were removed from the input, the performance decreased on each disease. Specifically, introducing the chief complaint can be most helpful to the diagnosis of pneumothorax, lung cancer, and pleural effusion, while the laboratory-test results affect the diagnosis of bronchiectasis and tuberculosis the most.

The impact of chief complaints and laboratory test results was investigated on each respiratory disease. Pneumothorax, pleural effusion, and lung cancer are the three pulmonary diseases whose diagnoses are affected most by chief complaints. In diagnosing pneumothorax, it was found that phrases like “chest pain” and “breath shortness” outweigh other terms. These findings are consistent with the guideline of the Mayo Clinic where a collapsed lung often leads to sudden chest pain and shortness of breath. Similarly, for pleural effusion, the unified model assigns ample attention to phrases like “pain worsens” and “when breathing”. “Coughing up blood” is the most typical symptom of lung cancer, as identified by the unified model. These results are broadly consistent with clinical experiences. On the other hand, tuberculosis and bronchiectasis are two pulmonary diseases whose diagnoses are primarily affected by laboratory tests. In the diagnosis of tuberculosis, it was found that adenosine deaminase is the most important among all laboratory test items. As for bronchiectasis, it was found that the test item “globulin” outweighs others.

### Example 5—Attention Visualization Results

FIGS. 3A-3F provide attention visualization results for a case with COPD. In FIG. 3A, the image modality (i.e., the radiograph) played a significant role in the diagnostic process, and its weight was nearly 80% in the final decision. Besides, the chief complaint was the second most important factor, accounting for roughly 16% weight. As FIG. 3B shows, PaO₂ (i.e., oxygen pressure in arterial blood) and PaCO₂ (i.e., partial pressure of carbon dioxide in arterial blood) were the two most important laboratory-test items, which are consistent with the observations reported in the literature 34. Nonetheless, that the total weight of the remaining 90 test items was quite large, whose distribution over these 90 laboratory-test items was nearly uniform. The reason might be that these laboratory-test items could help rule out other diseases. FIG. 3C shows that from the perspective of the unified model, age was a more critical factor than sex. FIG. 3D provides the attention map of the radiogra, implicating that the unified model would refer to hilar enlargement, hyper-expansion, and flattened diaphragm as the most important evidence for the diagnosis of COPD. Besides, the unified model could also identify large black areas due to bullae as relatively important evidence. FIG. 3E summarizes the experimental results with and without cross attention, where the sum of similarity scores of important (top 25%) tokens (i.e., words and image patches) are presented with the CLS token. With cross attention, the sum of similarity scores became larger, indicating that cross attention has improved the identification of important tokens compared to the model without cross attention. In FIG. 3F, the unified model recognized “sputum”, “dyspnea”, and “years” as the three most important words in the chief complaint, and provided the cross-attention maps between each of the top three important words and the image. The word “sputum” is primarily associated with the trachea and the lower pulmonary lobes in the image. The high attention area of the trachea could be reasonable because trachea was often the location where sputum might occur. The high attention region in the left lower lobe had reduced vascular markings, while both the left and right lower lobes of the lungs were hyperinflated. Hyperinflated lungs and reduced vascular markings are common symptoms of COPD, which often has abnormal sputum production. The model has also associated the word “dyspnea” with most areas of the lungs in the image because dyspnea can be caused by a variety of pulmonary abnormalities that could occur anywhere in the lungs. Lastly, the unified model disclosed herein has identified the areas surrounding the bronchi as the image regions associated with the word “years”, which implies “years” should be associated with chronic diseases, such as chronic bronchitis, which is often part of COPD.

### Example 6—Performance of a Unified Model Compared to Non-Unified Early and Late Fusion Paradigms

The unified model, as demonstrated in this example, is more effective than the previous non-unified early and late fusion paradigm in multimodal medical diagnosis. This is a prominent observation obtained from the experimental results, and it holds in both tasks of pulmonary disease identification and triage of COVID-19 patients. Specifically, the unified model outperforms previous early fusion and late fusion methods by an average of 9% and 10%, respectively, for identifying pulmonary diseases. Meanwhile, the unified model achieves about 3-percent performance gains on all eight diseases, and substantially improves the diagnostic performance on four diseases (i.e., bronchiectasis, pneumothorax, ILD, and tuberculosis) by boosting their AUROC by over 10%. These prominent performance benefits are closely related to several capabilities of the unified model. First, the unified model is built on top of a unified transformer MDT. MDT directly produces diagnostic decisions from multimodal input data, and learns holistic multimodal representations progressively and implicitly. In contrast, the traditional non-unified approach decomposes the diagnosis problem into several components, which, in most cases, consist of data structuralization, modality-specific model training, and diagnosis-oriented fusion. In practice, these components are hard to optimize and may prevent the model from learning holistic and diagnosis-oriented features. Second, inspired by physicians' daily activities, the unified model applies intra-and bi-directional inter-modal attention to tokenized multimodal data for exploiting the local interconnections among complementary modalities. On the contrary, the previous non-unified paradigm directly makes use of the extracted global modality-specific representations or predictions for diagnosis. In practice, the token-level attentional operations in proposed bi-directional multimodal attention help capture and encode the interconnections among the local patterns of different modalities into the fused representations. Last but not the least, the unified model is designed to conduct representation learning directly on unstructured raw texts. In contrast, the previous non-unified approach relies on non-clinically pre-trained NLP models to provide word embeddings, which inevitably distracts the diagnosis system from its intended functionality.

The superiority of the aforementioned abilities has been partly verified in the second task, i.e., adverse clinical outcome prediction of COVID-19 patients. From Table 2, the unified model holds a 7-percent average performance gain over the early fusion approach and an average of 9-percent advantage over the late fusion one. This performance gain is a little lower than that in the pulmonary disease identification task as there are no unstructured texts in the MMC dataset that the unified model can utilize. Nonetheless, the unified model can still leverage its unified and bi-directional multimodal attention mechanisms to better serve the goal of rapid triage of COVID-19 patients. For example, the unified model boosts the performance of MV and death prediction by 7% and 10%, respectively. Such substantial performance improvements brought by the unified model are valuable in the real world for allocating appropriate medical resources to patients in a timely manner, as medical resources are usually limited in the COVID-19 pandemic.

### The Unified Model Provides a Better Transformer-Based Choice for Jointly Interpreting Multimodal Clinical Information.

The unified model is compared to GIT³³ and Perceiver³⁰, two representative transformer-based models that fuse multimodal information for classification. GIT performs multimodal pre-training on tens of millions of image-text pairs by utilizing the common semantic information among different modalities as supervision signals. However, these characteristics have two obvious deficiencies in the medical diagnosis scenario. First, it is much harder to access multimodal medical data in the amount of the same order of magnitude. Second, multimodal data in the medical diagnosis scenario provide complementary instead of common semantic information. Thus, it is impractical to perform large-scale multimodal pre-training, as in GIT, using a limited amount of medical data. These deficiencies are also reflected in the experimental results. For instance, the average performance of GIT is about 7-and 8-percent lower than the unified model in the pulmonary disease identification task and adverse outcome prediction of COVID-19 task, respectively. These advantages show that token-level bi-direction multimodal attention in the unified model can effectively utilize limited amount of multimodal medical data and exploit complementary semantic information.

Perceiver simply concatenates multimodal input data and takes the resulting 1D sequence as the input instead of learning fused representations among modality-specific low-level embeddings as in the unified model. This poses a potential problem: the modality that makes up the majority of the input would have a larger impact on final diagnostic results. For example, since an image often has a much larger number of tokens than a text, Perceiver would inevitably assign more weight to the image instead of the text when making predictions. However, it is not always true that images play a more important role in daily clinical decisions. This point is also reflected in the experimental observations. For example, Perceiver yields clear performance improvements (2-percent gain on average in Table 1) over the early fusion model in identifying pulmonary diseases whereas the input radiograph serves as the main information source. But in the task of rapid triage of COVID-19 patients, the performance of Perceiver is only comparable to that of the early fusion method. The underlying reason is that CT images are not as helpful in this task as radiographs in pulmonary disease identification. In contrast, the unified model demonstrates satisfactory performance in both tasks by learning holistic multimodal representations through bi-directional multimodal attention. The method encourages features from different modalities to evenly blend into each other, which prevents the learned representations from being dominated by high-dimensional inputs.

### The Unified Model Helps Reduce the Reliance on Text Structuralization in the Traditional Workflow.

In traditional non-unified multimodal medical diagnosis methods, the usual way to deal with unstructured texts is text structuralization. Recent text structuralization pipelines in non-unified approaches¹⁹⁻²³ severely rely on artificial rules and the assistance of modem NLP tools. For example, text structuralization requires human annotators to manually define a list of alternate spellings, synonyms, and abbreviations for structured labels. On top of these preparations, specialized NLP tools are developed and applied to extract structured fields from unstructured texts. As a result, text structuralization steps are not only cumbersome but also costly in terms of labor and time. In comparison, the unified model abandons such tedious structuralization steps by directly accepting unstructured clinical texts as part of the input.

In conclusion, although NLP technologies particularly transformer have contributed significantly to latest AI diagnostic tools using either text-based electronic health records³⁵ or images³⁶, this study describes an AI framework consisting of a unified multimodal diagnostic transformer (MDT) and bi-directional multimodal attention blocks. This new algorithm enables the unified model to take a different approach from previous non-unified methods by progressively learning holistic representations for multimodal clinical data while eliminating separate paths for learning modality-specific features in non-unified techniques. This approach will be greatly enhanced by the latest development of large language models^{37,38}.

In real-world scenarios, the unified model may help streamline patient care, such as triaging patients and differentiating between those patients who are likely to have a common cold from those who need urgent intervention for a more severe condition. Furthermore, as the algorithms become increasingly refined, these frameworks could become a diagnostic aid for physicians and assist in cases of diagnostic uncertainty or complexity, thus not only mimicking physician reasoning but further enhancing it. The impact of the work may be most obvious in areas where there are few and uneven distributions of healthcare providers relative to the population.

To make the unified model adaptable to a changing environment, such as dealing with rapidly mutating SARS-CoV-2 viruses, the model can be trained on multiple cohorts jointly or resort to other machine learning technologies, such as online learning. Last but not the least, to address the problem of modal deficiency where one or more modalities may be unavailable, masked modeling²⁵ can be used. For instance, during the training stage, some modalities can be randomly masked to imitate the absence of these modalities in clinical workflows.

### REFERENCES

1. He, J. et al. The practical implementation of artificial intelligence technologies in medicine. Nature Medicine 25, 30-36, doi:10.1038/s41591-018-0307-0 (2019).
2. Liang, H. et al. Evaluation and accurate diagnoses of pediatric diseases using artificial intelligence. Nature Medicine 25, 433-438, doi:10.1038/s41591-018-0335-9 (2019).
3. Boehm, K. M., Khosravi, P., Vanguri, R., Gao, J. & Shah, S. P. Harnessing multimodal data integration to advance precision oncology. Nat. Rev. Cancer 22, 114-126 (2022).
4. Li, J., Shao, J., Wang, C. & Li, W. The epidemiology and therapeutic options for the COVID-19. Precis. Clin. Med. 3, 71-84 (2020).
5. Comfere, N. I. et al. Provider-to-provider communication in dermatology and implications of missing clinical information in skin biopsy requisition forms: a systematic review. Int. J Dermatol. 53, 549-557 (2014).
6. Shao, J. et al. Radiogenomic system for non-invasive identification of multiple actionable mutations and PD-L1 expression in non-small cell lung cancer based on CT images. Cancers (Basel) 14, 4823 (2022).
7. Huang, S. C., Pareek, A., Seyyedi, S., Banerjee, I. & Lungren, M. P. Fusion of medical imaging and electronic health records using deep learning: a systematic review and implementation guidelines. npj Digit. Med. 3, 136 (2020).
8. Wang, C. et al. Non-Invasive measurement using deep learning algorithm based on multi-source features fusion to predict PD-L1 expression and survival in NSCLC. Front. Immunol. 13, 828560 (2022).
9. Zhang, K. et al. Clinically applicable AI system for accurate diagnosis, quantitative measurements, and prognosis of COVID-19 pneumonia using computed tomography. Cell 181, 1423-1433.e1411 (2020).
10. Kermany, D. S. et al. Identifying Medical Diagnoses and Treatable Diseases by Image-Based Deep Learning. Cell 172, 1122-1131.e1129, doi:10.1016/j.cell.2018.02.010 (2018).
11. Rajpurkar, P., Chen, E., Banerjee, O. & Topol, E. J. AI in health and medicine. Nat. Med. 28, 31-38 (2022).
12. LeCun, Y., Bengio, Y. & Hinton, G. Deep learning. Nature 521, 436-444 (2015).
13. Schmidhuber, J. Deep learning in neural networks: an overview. Neural Netw. 61, 85-1173 (2015).
14. Wang, G. et al. A deep-learning pipeline for the diagnosis and discrimination of viral, non-viral and COVID-19 pneumonia from chest X-ray images. Nat. Biomed. Eng. 5, 509-521 (2021).
15. Zhou, H. Y. et al. Generalized radiograph representation learning via cross-supervision between images and free-text radiology reports. Nat. Mach. Intell. 4, 32-40 (2022).
16. Tang, Y. X. et al. Automated abnormality classification of chest radiographs using deep convolutional neural networks. npj Digit. Med. 3, 70 (2020).
17. Wang, C. et al. Development and validation of an abnormality-derived deep-learning diagnostic system for major respiratory diseases. npj Digit. Med. 5, 124 (2022).
18. Rajpurkar, P. et al. ChexNet: Radiologist-level pneumonia detection on chest x-rays with deep learning. arXiv preprint arXiv:1711.05225 (2017).
19. Mei, X. et al. Artificial intelligence-enabled rapid diagnosis of patients with COVID-19. Nat. Med. 26, 1224-1228 (2020).
20. Yala, A., Lehman, C., Schuster, T., Portnoi, T. & Barzilay, R. A deep learning mammography-based model for improved breast cancer risk prediction. Radiology 292, 60-66 (2019).
21. Zhang, K. et al. Deep-learning models for the detection and incidence prediction of chronic kidney disease and type 2 diabetes from retinal fundus images. Nat. Biomed. Eng. 5, 533-545 (2021).
22. Xu, Q. et al. AI-based analysis of CT images for rapid triage of COVID-19 patients. npj Digit. Med. 4, 75 (2021).
23. Akselrod-Ballin, A. et al. Predicting breast cancer by applying deep learning to linked health records and mammograms. Radiology 292, 331-342 (2019).
24. Vaswani, A. et al. Attention is all you need. Adv. Neural. Inf Process. Syst. 30 (2017).
25. Devlin, J., Chang, M.-W., Lee, K. & Toutanova, K. BERT: Pre-training of deep bidirectional transformers for language understanding. arXiv preprint arXiv:1810.04805 (2018).
26. Dosovitskiy, A. et al. An image is worth 16×16 words: Transformers for image recognition at scale. arXiv preprint arXiv:2010.11929 (2020).
27. LeCun, Y. et al. Handwritten digit recognition with a back-propagation network. Adv. Neural. Inf Process. Syst. 2 (1989).
28. Mikolov, T., Chen, K., Corrado, G. & Dean, J. Efficient estimation of word representations in vector space. arXiv preprint arXiv:1301.3781 (2013).
29. Mikolov, T., Sutskever, I., Chen, K., Corrado, G. S. & Dean, J. Distributed representations of words and phrases and their compositionality. Adv. Neural. Inf Process. Syst. 26 (2013).
30. Jaegle, A. et al. Perceiver: General perception with iterative attention. In Proc. 38th International Conference on Machine Learning 4651-4663 (2021).
31. Li, J. et al. Align before fuse: Vision and language representation learning with momentum distillation. Adv. Neural. Inf Process. Syst. 34, 9694-9705 (2021).
32. Su, W. et al. Vl-bert: Pre-training of generic visual-linguistic representations. arXiv preprint arXiv:1908.08530 (2019).
33. Wang, J. et al. GIT: A generative image-to-text transformer for vision and language. arXiv preprint arXiv: 2205.14100 (2022).
34. Pauwels, R. A., Buist, A. S., Calverley, P. M., Jenkins, C. R. & Hurd, S. S. Global strategy for the diagnosis, management, and prevention of chronic obstructive pulmonary disease. NHLBI/WHO Global Initiative for Chronic Obstructive Lung Disease (GOLD) Workshop summary. Am. J Respir. Crit. Care Med. 163, 1256-1276 (2001).
35. Li, Y. et al. BEHRT: Transformer for Electronic Health Records. Sci Rep 10, 7155, doi:10.1038/s41598-020-62922-y (2020).
36. Xia, K., Wang, J. Recent advances of transformers in medical image analysis: a comprehensive review. MedComm—Future Med. 2023; 2:e38 (2023)
37. Wang, D., Feng, L., Ye, J., Zou, J., Zheng, Y. Accelerating the integration of ChatGPT and other large-scale AI models into biomedical research and healthcare. MedComm—Future Med. 2023; 2:e43.
38. Moor, M. et al. Foundation models for generalist medical artificial intelligence. Nature 616, 259-265 (2023)
39. Ba, J. L., Kiros, J. R. & Hinton, G. E. Layer normalization. arXiv preprint arXiv:1607.06450 (2016).
40. Hendrycks, D. & Gimpel, K. Gaussian error linear units (gelus). arXiv preprint arXiv:1606.08415 (2016).
41. Srivastava, N., Hinton, G., Krizhevsky, A., Sutskever, I. & Salakhutdinov, R. Dropout: a simple way to prevent neural networks from overfitting. J. Mach. Learn. Res. 15, 1929-1958 (2014).
42. Johnson, A. E. et al. MIMIC-CXR, a de-identified publicly available database of chest radiographs with free-text reports. Scientific data 6, 1-8 (2019).
43. Jaegle, A. et al. Perceiver I O: A general architecture for structured inputs & outputs. arXiv preprint arXiv: 2107.14795 (2021).
44. Deng, J. et al. ImageNet: A large-scale hierarchical image database. In IEEE Conference on Computer Vision and Pattern Recognition 248-255 (2009).
45. He, K., Zhang, X., Ren, S. & Sun, J. Deep residual learning for image recognition. In IEEE Conference on Computer Vision and Pattern Recognition 770-778 (2016).
46. Ni, Q. et al. A deep learning approach to characterize 2019 coronavirus disease (COVID-19) pneumonia in chest CT images. Eur. Radiol. 30, 6517-6527 (2020).
47. Li, Z. et al. MVP-Net: multi-view FPN with position-aware attention for deep universal lesion detection. In International Conference on Medical Image Computing and Computer-Assisted Intervention 13-21 (2019).
48. Zhao, G. et al. Diagnose Like a Radiologist: Hybrid Neuro-Probabilistic Reasoning for Attribute-Based Medical Image Diagnosis. In IEEE Trans. Pattern Anal. Mach. Intell. 44, 7400-7416 (2021).
49. Loshchilov, I. & Hutter, F. Decoupled weight decay regularization. arXiv preprint arXiv:1711.05101 (2017).
50. Paszke, A. et al. Pytorch: An imperative style, high-performance deep learning library. Adv. Neural. Inf Process. Syst. 32 (2019).
51. Micikevicius, P. et al. Mixed precision training. arXiv preprint arXiv:1710.03740 (2017).
52. Selvaraju, R. R. et al. Grad-cam: Visual explanations from deep networks via gradient-based localization. In IEEE International Conference on Computer Vision 618-626 (2017).

The present disclosure is not intended to be limited in scope to the particular disclosed embodiments, which are provided, for example, to illustrate various aspects of the present disclosure. Various modifications to the compositions and methods described will become apparent from the description and teachings herein. Such variations may be practiced without departing from the true scope and spirit of the disclosure and are intended to fall within the scope of the present disclosure.

1. A method of training a model for providing a medical diagnosis, wherein the model comprises a free-form embedding layer, an image embedding layer, a plurality of bidirectional multimodal attention blocks, a self-attention block, and a classification head, the method comprising:
(a) obtaining multimodal input data of a subject, wherein the multimodal input data comprise a set of medical images and a set of clinical text data;
(b) tokenizing and embedding the set of medical images using the image embedding layer to generate a sequence of image patch tokens; and tokenizing and embedding the set of clinical text data using the free-form embedding layer to generate a sequence of clinical text tokens;
(c) passing the sequence of clinical text tokens and the sequence of image patch tokens to the plurality of bidirectional multimodal attention blocks to generate a bag of unified tokens,
wherein each of the plurality of bidirectional multimodal attention blocks applies (i) a first attention to capture intramodal connections in the set of clinical text data, (ii) a second attention to capture intramodal connections in the set of medical images, and (iii) a third attention to capture intermodal connections between the set of clinical text data and the set of medical images, and
wherein both the intramodal connections and the intermodal connections are encoded into latent representations, and representations within the same modality and across multiple modalities are learned and fused simultaneously; and
(d) passing the bag of unified tokens to the self-attention block to learn a holistic multimodal representation of medical diagnosis for inputting to the classification head, thereby training the model for providing the medical diagnosis.

2. The method of claim 1, wherein the free-form embedding layer is configured to convert unstructured and structured texts into uniform text tokens.

3. The method of claim 1, wherein the free-form embedding layer is configured to convert a chief complaint, a laboratory test result, and/or a demographic information into clinical text tokens.

4. The method of claim 1, wherein the image embedding layer comprises a convolutional layer configured to produce a sequence of visual tokens.

5. The method of claim 1, wherein the model comprises two stacked bidirectional multimodal attention blocks.

6. The method of claim 1, wherein each of the plurality of bidirectional multimodal attention blocks comprises two-layer normalization layers, a bidirectional multimodal attention layer, and a multilayer perceptron.

7. The method of claim 1, wherein the model comprises stacked self-attention blocks.

8. The method of claim 7, wherein each self-attention block comprises two-layer normalization layers, a self-attention layer, and a multilayer perceptron.

9. The method of claim 1, wherein the classification head is configured to identify a disease or condition and/or predict an adverse clinical outcome in a patient.

10. The method of claim 1, wherein the set of clinical text data comprises a chief complaint, a demographic information, and a laboratory test report.

11. The method of claim 10, wherein the chief complaint comprises unstructured data.

12. The method of claim 10, wherein the chief complaint comprises structured data.

13. The method of claim 1, wherein the set of clinical text data comprises: an unstructured chief complaint comprising a history of present and past illness; a laboratory test report comprising a plurality of test results; gender; and age.

14. The method of claim 1, wherein the set of clinical text data comprises: an structured chief complaint comprising comorbidities and symptoms; a laboratory test report comprising a plurality of test results; gender; and age.

15. The method of claim 1, wherein the set of clinical text data comprises multiple categories of clinical text data, and the method comprises concatenating encoded feature vectors for each category of clinical text data to produce the sequence of clinical text tokens.

16. The method of claim 1, wherein the set of medical images comprises one or more CT images, one or more X-ray images, or a combination thereof.

17. The method of claim 1, wherein each of the plurality of bidirectional multimodal attention blocks comprises multiple heads configured to perform attention operations in multiple representation subspaces simultaneously and aggregate results from the multiple representation subspaces afterwards.

18. The method of claim 1, wherein the self-attention block comprises multiple heads configured to perform attention operations in multiple representation subspaces simultaneously and aggregate results from the multiple representation subspaces afterwards.

19. The method of claim 1, comprising applying average pooling to the bag of unified tokens generated from the self-attention block.

20. A method of generating a medical diagnosis for a subject, the method comprising:
receiving a prompt for obtaining the medical diagnosis and a set of data related to the subject, and
generating the medical diagnosis by inputting the prompt and the set of data in a trained model generated by the method of claim 1.
